SEQUENCE LISTING (79 sequences)

SEQ ID NO: 1 - protein, 299 aa, Trichoderma reesei
LAILDKPVIQPNFPGNGLASLGQGLMDHLAPTHSTWDDWGAGWIPQDCKSLVEGAGLSASDVIPFNIHYDDCAQTWTFCRHKDSPLSEITILDIFGRLPVRMRSFVRHLVFIPGAKSAGSNGDNVLLRGDIDVTVFAHEIGHSLDSHAFDPSYGVPFSTGNVWISNYNLDSAVPDPYAQTSQQENFAQQTVISLYDKVVPGGIGTIQPNWQAIFHQYATLEGYIGDVIIPGGTCTHRLTDSPPVQMSSLAKGRRALGPKPNVALSPEVEEIEAIPMKSSITMTSFDEAGKPNGTYVINL

SEQ ID NO: 2 - protein, 298 aa, Cordyceps confragosa
ALNKPIINPQFPNGGLGSLGPGLMQHLQPAKRTWDKWGPGWIPEDCKNLAKSNNFSPFDITPFNIHYEDCPDSWVFCRHKDSPLSETAMIDMFGRMPVKMRSYIRHIIALPGTRSAGSAGDNIQMNGDCEITVFVHETGHSLDSHAFDPKYGAPFSNSKVWLDNYNQDSAVCDGYAQSSQQENLAQETVVGLFDKVVPGGVGTIQPNWKAIFHQYATVQGYLGDQILPGGTCSHRLTNSKPVPMGDSKKSRRGLGPAPDVSLSAHVTEIEPIAMGDSIVLTQYNDGGDAISKREVTLN

SEQ ID NO: 3 - protein, 299 aa, Torrubiella hemipterigena
RLDKPIINPTFPGGGLDSLAQGLLDHLAPTQSTHDNWGAGYIPEDCKNLAKSNGFSPFDITPFNIHYSDCNDVWIFCRHKDSPLSQIDMIDLFGRMPVHMREYIRHVIALPGNKSAGSSGDNIQMNGAVGITVFVHETGHSLDSHGFDPKYGVPFSNSKTWLDNYNMDTAVCDSYAQSSQQENLAQETVVGLYDKVVPGGVGNIQPNWKAIFHQYATVQGYLGDILTPGGTCRHRLANSKPVPMGDSAKFRRDLGPAPDVSLSSDVIEIESIPMGSSIRITEFDEAGNPIGSKNVSLVM

SEQ ID NO: 4 - protein, 297 aa, Trichoderma longibrachiatum
ILDKPVIQPNFPGNGLASLGQGLMDNLSPTHSTWDDWGAGWIPQDCKSLVEGAGFSASDVIPFNIHYDDCAQTWTFCRHKDSPLSEITILDIFGRLPVRMRSFVRHLVFIPGNKSAGSSGDNVLMVGAVDVTVFAHEIGHSLDSHAFDPSYGVPFSTGNVWISNYNLDSAVTDPYAQTSQQENFAQETVVSLYDKVVPGGIGTIQPNWQAIYHQYATLEGYIGDVIIPGGTCTHRLTDSPPVQMSSSSKLRRALGPKPHVALSPEVEEIEAIPMKSSITMTSFDAAGKPNGTYVINL

SEQ ID NO: 5 - protein, 297 aa, Trichoderma citrinoviride
TLDKPVIQPNFPGNGLASLGQGLLDNLAPTHSTWDDWGAGWIPQDCKTLVEGAGFSASDVIPFNIHYDDCAQTWTFCRHKDSPLSEVQILDLFGRLPVRMRSFVRHLVFIPGNKSAGSSGDNVLMTGPVDITVYAHEIGHSLDSHAFDPSYGVPFSTGNVWISNYNLDSAVTDPYAQTSQQENFAQETVVSLYDKVVPGGIGTIQSNWQAIFHQYATLEGYIGDVIIPGGTCTHRLTDSPAVPMSGSAKLRRGLGPKPHVALSPEVEEIEAIPMKSSITMTSFDEAGKPNGTYVINL

SEQ ID NO: 6 - protein, 297 aa, Trichoderma harzianum
NLNKPVITPNFPGNGLASLGPGLMNNLAPTHSTWDDWGAGWIPSDCKSMVQGAGFSPNDIIPFNIHYDDCAQTWTFCRHKNSPLSEIQILDIFGRLPVRMRQFVRHLVFVPGNKSAGSNGDNVLMVGDVDVTVFAHEIGHSLDSHAFDPSYGVPFSTGNVWVSNYNLDSAVPDPYAQSSQQENFAQQTVVSLYDKVVPGGIGTIQPNWQAIFHQYATLEGYIGNVIIPGGTCTHRLTDSAPVTMSGSKKSRRALGPKPKVSLSPEVKEIEAIPMESSITMTSFDEHGKPNGTYVIHL

SEQ ID NO: 7 - protein, 297 aa, Trichoderma guizhouense
NLNKPVITPNFPGNGLASLGPGLMNNLAPTHSTWDDWGAGWIPADCKSMVQGAGFSPNDIIPFNIHYDDCAQSWTFCRHKNSPLSEIQILDIFGRLPVRMRQFVRHLVFVPGNKSAGSNGDNVLMVGDVDVTVFAHEIGHSLDSHAFDPSYGVPFSTGNVWVSNYNLDSAVPDPYAQSSQQENFAQQTVVSIYDKVVPGGIGTIQPNWQAIFHQYATLEGYIGNVIIPGGTCTHRLTDSAPVTMSGSKKSRRALGPKPKVSLSPKVKEIEAIPMESSITMTSFDEHGKPNGTYVVHL

SEQ ID NO: 8 - protein, 297 aa, Trichoderma atroviride
KLDKPVIQPNFPGGGLASLGQGLLDNLKPTQSTSDVWGAGWIPADCKSLVQGAGLSPNDITPFNIHYTDCSQTWTFCRHKDSPLSQTDIIDLFGRVPVRMRDYIRHYVFIPGNKSAGSSGDNTLMVGDVDITVFLHEIGHSLDSHAFDPSYGVPFSTGNVWISNYNQDSAVTDDYAQSSQQENFAQETVVSVYDKNVPGGIGNIQPNWQAIFHQYATLEGYIGDTIIPGGTCVNRLTDSQPVTMSNSAKFRRNLGPKPTVSLSPDVKEIKAIPMADSIKMTSFDEHGKPNGTYVIQL

SEQ ID NO: 9 - protein, 294 aa, Trichoderma arundinaceum
NLNKPVIQPNFPGNGLASLGQGILDHLAPTQSTWDDWGAGWIPQDCKSLAQGAGLSPNDIIPFNIHYADCAQTWTFCRHKNSPLSEVNIIDLFGRLPVRMRQFVRHLVFIPGNKSAGSNGDNVLMVGPVDITVFAHEIGHSLDSHAFDPSYGVPFSTGNVWVSNYNLDSAVPDPYAQSSQQENFAQHTVISIYDKAVPGGIGTIQPNWQAIFHQYATLEGYIGDIIIPGGTCTHRLTDSPPVTMASKRRALGPKPKVALSPEVEEIEAIPMKNTITMTSFDEAGKPNGSYVINL

SEQ ID NO: 10 - protein, 293 aa, Trichoderma harzianum
NLDKPVIQPNFPSLAPGLMSHLAPTHSTWDDWGAGWIPQDCKSMVQGAGFSPNDIIPFNIHYDDCGQTWTFCRHKNSPLSELQILDMFGRLPVRMRSFVRHLVFVPGNRSAGSNGDNILVVGDGDITLFAHEIGHSLDSHAFDPSYGVPFSTGNVWVSNYNLDSAVSDSYAQTSQQENFAQETVISLFDKVVPGGIGTIQPNWQAIFHQYATLQGYIGNVIIPGGTCTHRLTDSPPVPMSGSAKSRRGLGPKPEVSLSPEVKEIEAIPMESSITMTSFDEHGKPNGTYVIHLK

SEQ ID NO: 11 - protein, 280 aa, Cordyceps confragosa
RLDKPVIKPPFPNGGLGSLGPDLMSSLKVASHTTDDWDKNHLPRDCKSLAQNAGFSPLDVSAFNIHYDDCKSQPWVFCRHKDSPLSKKDMTELFGRMPVRMRQFIRHIIALPGQRSAGSSGDNIQMNGDVEITVFIHETGHSLDSHAFDPKLGAPFSNGKAWTDAYNKDSAVTDDYAQTSQQENFAQETVVSVYDKNVKGGIGKIQPNAQAIRNAYTTLEKYIGNIIIPGGVCTHRLENSPSVPKADSARTRLVPKSEPVGEGLTRIEPIAMGPITPASY

SEQ ID NO: 12 - protein, 280 aa, Cordyceps fumosorosea
TLDKPVIKPPFPNGGLGSLGPDLMSSLPIAAHTTTDWDADHLPRDCKTLAQGAGFSPLDVSAFDIHYDDCKAQPWVFCRHKDSPLSKTDMTELFGRLPVRMRQFIRHIIALPGQRSAGSAGDNIQMNGDCEITVFIHETGHSLDSHAFDPKLGVPFSNGKAWTDAYHKDTAVTDDYGRTSQQENFAQETVVSVYDKNVEGGIGKIQPNAQAIRNAYTTLETYIGNIIIPGGECTHRLENSPSVPKADSARTRLVARAEPVGEGLTRIEPVPFGPILSVRY

SEQ ID NO: 13 - protein, 282 aa, Beauveria bassiana
RLDKPVIKPPFPNGGGLGSLGPDLMSSLPIAAHTTSDWDSDHLPRDCKRLAQSAGFSPLDVDAFNIRFDDCKQQPWVFCRHKDAPLSKSNMTELFGRLPVRMRQYIRHIIALPGQRSAGSAGDNIQMNGDCEITVFIHETGHSLDSHAFDPKHGVPFSNSPVWKDAYDKDTAVTDDYAQTSQQENFAQETVVAVYDKNVKGGGIGKIQPNAQAIRNAYTTLEKYIGDIIIPGGECTHRLENSPAVPKVDSARTRLVAKSEPVGEGLTRIEPIPMGPITLARY

SEQ ID NO: 14 - protein, 289 aa, Cordyceps fumosorosea
GSPDKPEMNPAFDYGRFEAGLRDNLHPTHSTWDYWGPGWIPQSCKDIATSHGLSPNDFTIFNVHYDDCQEGWTFCRHKDAGASEIDMIDIFGRAPVHMRSFIRHLVATPGLHDGAAAFTFSNGDCVVESAPDLSVMLHEMSHSLDWHALPQYPSPFSSSNEWQSNYAQDSNTPTAYGRTNWLEDFAEAGKVGVFDKVVPGGFGSVAGNWGQVFHQYATYQGYLGDTILPGGTCDKRFPNSETVPMAGNSKRMAADLGEKPDNTIKADNMTLIQMRPEIEGQVSICEHQH

SEQ ID NO: 15 - protein, 298 aa, Acremonium chrysogenum
QLDKPVIDPEFPSGALDQGLNDHLRAPGSSWDAWEEGWIPEDCKNMIEGTEFSVQDITPFNIHYDDCEDPWVFCRHKDSPLSEDYIVDAFGKMPVRFRSFVRHMLFVPGTESAGSSGDNVQMNGDLPITVFIHETAHSFDGHHGLPDIEGGFSASQAWLDAYNADSAVTDDYGQTSQAENFAQQTVVALFDSNVPDGVASIQPSWQSIENQFRLIQDRAADILRPDGTCTSRLENSAPVEKGGSARLRPSASTKPDVSLKGKIKVIEPVPMGAEMEMAQYDASGKVVGTKVVKVGGRE

SEQ ID NO: 16 - protein, 284 aa, Aspergillus undulatus
QLDKPALTDNLDYLLDGNVANLPSNGGPYWGKWEDGLIPADCKSIAESQGLNPTDFEVYDIFFDDCQDGWSFCRHKDSSDSFETLVDTFGRVPVRMRSWVRHILTIPGNNWAFNSNGNIAFSGTTSSNLDVALHETGHSLDLLGAYGDGALSSSQEWEDAYNADSNVPDNYAQSNQVENVAQNTVVGIYDKVVPGGFPGVQPNYANIQNQYTLLQDKAGEQIIPGGTCDRHLENSDTVSMSGSSSKRDVRAAKARAASHDSALKGKYSNIVKDFKPFNSQDFHH

SEQ ID NO: 17 - protein, 290 aa, Aspergillus recurvatus
DDTLSKPALTDNLDYLLDGNVASLPSNGGPYWGKWEDGLMPADCKSIAEDQGLNPTDFEVWDIFFDDCQDGWSFCRHKDSADSFETLVDTFGRVPVRMRSWVRHVLTIPGDNWAFNSNGNICFSGTTSQNLDVALHETGHSLDLLGAYGDGALSSSQEWLDAYNADSNVPDNYARSNQVENVAQNTVVAVYDKNVPGGFPGVQTDYAKIQNQYTLVEDKAGDQIVPGGTCDRHLENSETVPMSGSSAKRDIRIKGRAVGAGAVTHDWKFKGTYSNIVTDFEPFNSKDFHH

SEQ ID NO: 18 - protein, 302 aa, Exidia glandulosa
QNLGKPVLFTDGLSPHVDASFWAHLNPTQSTWDQWGWGWIPQSCFDAANSNNVSPYDFEVFNVHYTDCGTAWVFCRHHSASLSQIDMIDLFGRLPVHERQWIRHVVAVPGGGSAYELNADVVFQGPVGTPSVFQHEVGHAVDFYKNGFQSSGTSQFLNAIQQDSCVPDDYANSNNVEDYTQVGVLSLYEIVNPGGLDPIGANWRCLVNQKNVLDGFQRDNMIPGGVCDRRWADSTIVKMGPAVPRVKRGPVGPIPQPAGLPVPGPSKAGPDVYKNLVFNATEKRNAETRQAAWQKAAKGAEA

SEQ ID NO: 19 - protein, 279 aa, Rasamsonia emersonii
QRLNKPALTPNLDYLEQGNIDNLHPTHSTWDKWGPGWIPADCKSIAQNEGKNPDDFEIYNVHYDDCSDAWVFCRHKDSNVDLVTTIDIFGRLPVKMRSWVRHVITIPGDSWAYNSNGNIAFFGTTSSSMDVAIHETGHSLDLLGAYWGKTFSDSSTWLDNYNQDPNVPDEYAQTNQVENVAQNTVVGVYDKVVPGGFGSVQPNWHNIFHQYATLQWGAGDQIIPGGTCDRHLQNSETVPTGNSALAFNAVNRPDTSLKGNYSNIVTDFKEFSTRGNCRI

SEQ ID NO: 20 - protein, 286 aa, Aspergillus rambellii
QTLDKPALTPNLDYLLQGNTDNLQPAASHYGKWEDGWIPADCKSIAEDQQLNPTDFEVYDVFYGDCGDAWSFCRHKSSSDSIDTIIDTFGRVPVRMRSWVRHVLTIPGDNWAFNSNGNIAFSGTTSQNLDVALHETGHSLDLLGAYGETLSSSQTWLDNYNQDPNVPDEYARSNQVENVAQNTVVGIYDKVVPGGFGSVQPNWGNIFHQYSTLQSMAKDTIIPGGTCDRHLANSETVSMASTQSNATRMARIRRVKPSHSFRQTYDNIVEGFTPFSTKDTCHFSWA

SEQ ID NO: 21 - protein, 283 aa, Aspergillus versicolor
LDKPALTPDLDYLLDGNTANLPQNGGAHWGKWEDGLMPEDCKSIAEGQDLNPADFEVYDVFYDDCQDAWSFCRHKDSPEDLETLVNTFGRVPVRMRSWVRHVLSIPGNNWAFNSNGNIALSGTTTHNLDVCLHETGHSLDLLGAYVDGALSSTQEWEDAYNKDSAVPDNYARTNQVENVAQNTVVGIYDKVVPGGFPGVQSESQKIENQYKLLQDKAGEQILPGGTCDRHLQNSATVSIKSSSKRENKVVKAREAHDSALKGTYTNIVKEFTPFNTKDFHEEH

SEQ ID NO: 22 - protein, 284 aa, Aspergillus bisporus
QLDKPALTSNLDYLLEGNTNNLPRNGGPYWGKWADGLMPADCKSIAESQGLNPTDFEVWDIFYNDCQDGWSFCRHKDSSDSFETLVETFGRVPVRMRSWVRHILTIPGPNWAFNSNGNIALSGTTSSNLDVALHETAHSLDLLGAYGNGALSSMQEWLNAYNRDSHVPDEYARSNQVENVAQNTVVAIYDKVVPGGFPGIQPNYQRIQNQYTLVQAKAGNQILPGGTCDRHLENSDTVPMSGLSLAYKLDSEKSRGVWHDSAFKGTYMNIVKEFTPFNTQEFHH

SEQ ID NO: 23 - protein, 286 aa, Aspergillus amylovorus
QQLDKPALTNDLDYLLEGNVNNLPSNGGPYWGKWEAGLMPADCKSIAESQDLDPTDFEVWDIFFDDCQDGWSFCRHVDSADSFETLVDTFGRIPVRMRSWIRHILTIPGDNWAFNSNGNIAFSGTTSANLDVALHETGHSLDLLGAYGSEALSSTQEWLDAYDADSNVPDNYARSNQVENVAQNTVVGIYDKVVPGGFPGVQPNYQNIENQYTLLQDKAGDQIIPGGTCDRHLENSETVSASGSSKKARDSVNKKSRAVAHDPAFKGTYTNIVKDFVPFNTQDYHH

SEQ ID NO: 24 - protein, 297 aa, Trichoderma asperellum
KLDKPIIQPNFPGGGLASLGQGLLNNLSPTQSTSDNWGAGWIPADCKSLVQGAGLSPFDVTPFNIHYTDCSQTWTFCRHKDSPLSQTDIIDLFGRVPVRMRDFIRHYVFIPGATSAGSSGDNTLMVGNVDITVFLHEIGHSLDSHAFDPSYGVPFSTSNVWISNYNLDSAVTDSYAQTSQQENFAQETVVSVYDKVVPGGIGTIQPNWQAIFHQYATLQGYIGNTIIPGGTCVNRLTDSQPVTMGNSAKFRRGLGPKPTVSLSPDVKEIKPIPMSDSITMTTFDEFGKANGTYVIHL

SEQ ID NO: 25 - protein, 285 aa, Trichoderma asperellum
GNPDKPEVNPHIPYDRLENDLRNNLHPTQSTWDYWGAGWIPQGCKDLANTYHLNPADFTIFNVHYTDCSASWTFCRHKNAGASEIDMIDIFGRLPVRMRSYLRHLVATPGLNDGAAAWTWSSGDCVVESAPNLATLVHEVSHSLDWHALPQYQQPFSSSSIWQDNYNQDSATPTGYGRTNWMENFAETGMVGVYDKAVPGGIGNIEPNWNAIFHQYATYQGYLGDKIIAGGSCANRFDDTPPVAMSNSAKFKITSAKPETGIDFAGKNITQIIPSKEFEGLYIKG

SEQ ID NO: 26 - protein, 336 aa, Trichoderma virens
QNLNKPVLFSDGLSPHVDSVFFQHLTPTQSTWDKWGWGWIPQACLSVVQGTQFSPYDIEVYNVHYTDCASAVVMCRHNQAQMSIINMIDAFGRIPIHDRQWTQHILALPAGGCSAGTGGAVTTFYGPCYIPSVFVHELTHTLDAFVNDKTGSTGAVHVYSTTSSYLNSVNQDSCVPDGYANTSPQEDYAQTSVLALYEKVNPGGLDPIGPWRCLVNAKNNLDSIIGQELTPGGSCTHRWADSAIVCMGPAAGCTSSKRGFEGRRGLKPVGPKPNPANKKGDPLVLDLLPDLASVVNAKITDFSHLSSNHTAGIEATIRINAWNKAAGKETVPVPKA

SEQ ID NO: 27 - protein, 397 aa, Trichoderma reesei
QQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 28 - protein, 383 aa, Fungal sp.
QQTAWGQCGGQGWTGATTCVSGYYCSFQNNWYSQCLPGAASTTSTSTTTLKTTTTTSSTSSAPTGKVRFAGVNIAGFDFGVVTSGTQDLSQVVDESVDGVNQMSHFVNADTFNIFRLPTGWQFIVNNNLGGPLDSNNFGKYDKLVQGCLSLGAYCIVDVHNYARWNGGVIGQGGPTDDQFISLWTQLATHYKSNSKVIFGIMNEPHDLNIATWAATVQKTVTAIRNTGATSQMILLPGTDYTSAANFIENGSGAALLPVKNPDGSTTNLIFDVHKYLDSDNSGTHAECVTNNADAFNNLATWLRSNKRQALLSETGGGNVQSCATYMCQQLDILNANSDVYLGWTSWSAGGFQASWNYVLTEVPVNGVDQTLVQQCFVPKWKN

SEQ ID NO: 29 - protein, 440 aa, Aspergillus flavus
QQVGTYQTETHPSMTWQTCGNGGSCSTNQGSVVLDANWRWVHQTGSSSNCYTGNKWDTSYCSTNDACAQKCALDGADYSNTYGITTSGSEVRLNFVTSNSNGKNVGSRVYMMADDTHYEVYKLLNQEFTFDVDVSKLPCGLNGALYFVVMDADGGVSKYPNNKAGAKYGTGYCDSQCPRDLKFIQGQANVEGWVSSTNNANTGTGNHGSCCAELDIWESNSISQALTPHPCDTPTNTLCTGDACGGTYSSDRYSGTCDPDGCDFNPYRVGNTTFYGPGKTIDTNKPITVVTQFITDDGTSSGTLSEIKRFYVQDGVTYPQPSADVSGLSGNTINSEYCTAENTLFEGSGSFAKHGGLAGMGEAMSTGMVLVMSLWDDYYANMLWLDSNYPTNESTSKPGVARGTCSTSSGVPSEVEASNPSAYVAYSNIKVGPIGSTFKS

SEQ ID NO: 30 - protein, 509 aa, Neosartorya fischeri
QQIGTYTTETHPSMTWETCTSGGSCATNQGSVVMDANWRWVHQVGSTTNCYTGNTWDTSICDTDETCATECAVDGADYESTYGVTTSGSQIRLNFVTQNSNGANVGSRLYMMADNTHYQMFKLLNQEFTFDVDVSNLPCGLNGALYFVTMDEDGGVSKYPNNKAGAQYGVGYCDSQCPRDLKFIQGQANVEGWTPSSNNENTGLGNYGSCCAELDIWESNSISQALTPHPCDTATNTMCTGDACGGTYSSDRYAGTCDPDGCDFNPYRMGNTTFYGPGKTIDTNSPFTVVTQFITDDGTDTGTLSEIRRYYVQNGVTYAQPDSDISGITGNAINADYCTAENTVFDGPGTFAKHGGFSAMSEAMSTGMVLVMSLWDDYYADMLWLDSTYPTNASSSTPGAVRGSCSTDSGVPATIESESPDSYVTYSNIKVGPIGSTFSSGSGSGSSGSGSSGSASTSTTSTKTTAATSTSTAVAQHYSQCGGQDWTGPTTCVSPYTCQVQNAYYSQCL

SEQ ID NO: 31 - protein, 431 aa, Chaetosartorya sp.
QQTVWGQCGGQGWSGPTNCAAGSACSTLNPYYAQCIPGATTTSSTLSTTTKTTTTTTTGPTGPTTSPPTVTASGNPFSGYQLYANPYYSSEVNTLAMPSLPSSLQPKASAVAKVPSFVWLDVAAKVPTMGTYLADIQAKNKAGANPPIAGIFVVYDLPDRDCAALASNGEFSIANNGVANYKAYIDSIRAQLVKYSDVHTILVIEPDSLANLVTNLNVQKCANAQSAYLECVNYALKQLNLPNVAMYIDAGHAGWLGWPANLGPAATLFAKVYTDAGSPAAVRGLATNVANYNAWSLSTCPSYTQGDSNCDEKKYVNAIAPLLQQAGFNAHFITDTSRNGVQPTKQNAWGDWCNVIGTGFGVRPTTNTGDPLEDAFVWVKPGGESDGTSNSSSARYDAHCGYSDALQPAPEAGTWFQAYFQQLLTNANPSF

SEQ ID NO: 32 - protein, 440 aa, Rasamsonia composticola
QQSLWGQCGGSSWTGATSCAAGATCSTINPYYAQCVPATATPTTLTTTTKPTSTGGAAPTTPPPTTTGTTTSPVVTRPASASGNPFEGYQLYANPYYASEVISLAIPSLSSELVPKASEVAKVPSFVWLDQAAKVPSMGDYLKDIQSQNAAGADPPIAGIFVVYDLPDRDCAAAASNGEFSIANNGVALYKQYIDSIREQLTTYSDVHTILVIEPDSLANLVTNLNVPKCANAQDAYLECINYAITQLDLPNVAMYLDAGHAGWLGWQANLAPAAQLFASVYKNASSPASVRGLATNVANYNAWSISPCPSYTQGDANCDEEDYVNALGPLLQEQGFPAYFITDTSRNGVQPTKQSQWGDWCNVIGTGFGVRPTTDTGNPLEDAFVWVKPGGESDGTSNTTSPRYDYHCGLSDALQPAPEAGTWFQAYFEQLLTNANPPF

SEQ ID NO: 33 - protein, 263 aa, Penicillium funiculosum
PAAEPVSFDIRDENITLARRAEAINYNQDYIASGANVQYSPNMAAGSFSINYNTQGDFVVGLGWQPGDANPITYSGSFSASGVGILAVYGWSTNPLVEYYVMEVHDGYQTAGTHKGTVTTDGGTYDIWEHQQVNQPSILGTSTFNQYISIRQSPRTSGTVTVQNHFNAWAQAGMNLGTLNYQVMAVESWSGSGSGQISLSKGTGGGSTTTTPTGPTSTSTAPSSGGTGAAQWGQCGGIGWTGPTTCVAPYTCKYENAYYSQCQ

SEQ ID NO: 34 - protein, 559 aa, Paraphaeosphaeria sporulosa
QTSVDTFVTSQRSVSIQGVLSNIGADGSKAQGAAAGIVVASPSKSNPDYWYTWTRDSALTYKALVERFIGGDTTLRKKLDEYVSSQAYLQTVSNPSGGPDTGGLGEPKFNVDRTAFTGAWGRPQRDGPPLRATALTLYANWLIANGNTTQAANTLWPVIAKDLAYAVKYWNQTGFDLWEEVNGSSFFTLAATHRALVEGAALATKLSKTCDGCAAAAPQILCFLQSFWTGSYIDSNINVNDGRTGKDVNSIISSIHTFDPASACTDATFQPCSSRALLNHKAVTDSFRTVYGINKGIAQGQAVAVGRYSEDVYYNGNPWYLATLAAAEQLYAAVYQWNTIGSITVDSVSLPFFKDLLPSIATGTYASSSATFASIVSAVKTYGDGYVAVVQKYTPANGGLAEQFDKSSGSPLSAVDLTWSYAAFLTATDRRSGSVGPSWGEKSNNVPPTSCTAPPSCNVQVTFNERVTTAYGDNIFIVGQLTQLGNWDPNSAVALSASKYTSSDPLWYATVSLPASTSFAYKYIKKTSSGTVVWESDPNRSYTTATTCGSTATQNDTWR

SEQ ID NO: 35 - protein, 599 aa, Paraphaeosphaeria sporulosa
QTSVDTFVTSQRSVSIQGVLSNIGADGSKAQGAAAGIVVASPSKSNPDYWYTWTRDSALTYKALVERFIGGDTTLRKKLDEYVSSQAYLQTVSNPSGGPDTGGLGEPKFNVDRTAFTGAWGRPQRDGPPLRATALTLYANWLIANGNTTQAANTLWPVIAKDLAYAVKYWNQTGFDLWEEVNGSSFFTLAATHRALVEGAALATKLSKTCDGCAAAAPQILCFLQSFWTGSYIDSNINVNDGRTGKDVNSIISSIHTFDPASACTDATFQPCSSRALLNHKAVTDSFRTVYGINKGIAQGQAVAVGRYSEDVYYNGNPWYLATLAAAEQLYAAVYQWNTIGSITVDSVSLPFFKDLLPSIATGTYASSSATFASIVSAVKTYGDGYVAVVQKYTPANGGLAEQFDKSSGSPLSAVDLTWSYAAFLTATDRRSGSVGPSWGEKSNNVPPTSCSMGSATGTYSTATATSWPSTLTSGSPGSTTTVGTTTSTTSGTAAETAVQVTFNERVTTAYGDNIFIVGQLTQLGNWDPNSAVALSASKYTSSDPLWYATVSLPASTSFAYKYIKKTSSGTVVWESDPNRSYTTATTCGSTATQNDTWR

SEQ ID NO: 36 - protein, 299 aa, Niesslia exilis
ALDKPIIDPEFPGGGLASLGQGLLDNLAPTQSTYADWEAGFIPQDCKTIAEGNSLSPLDIIPFNITYTDCSDPWIFCRHKDSPLSEVDMIDLFGRMPVGMRSFIRHIISIPGDKSAGSNGDNVQMNGDVDITVFIHETGHSLDSHGFDASYGVPFSTGQVWTDNYNLDSAVTDSYAQSSQQENFAQETVVALYDKVVPGGVGTIQPNWQAIQHQYETIQGYAGGIIIPGGTCTNRLPNSPAVPMSDSAKYRVNMAHKPDVSLSNYVTEIKSIPMEPIMKLTHYDAAGRPIGKSTIDLST

SEQ ID NO: 37 - protein, 297 aa, Trichoderma harzianum
NLNKPVITPNFPGNGLASLGQGLMDHLAPTHSTWDDWGAGWIPQDCKSMVQGAGFSPNDIIPFNIHYDDCAQTWTFCRHKNSPLSEIQILDIFGRLPVRMRQFVRHLVFVPGNKSAGSNGDNVLMVGDVDVTVFAHEIGHSLDSHAFDPSYGVPFSTGNVWVSNYNLDSAVPDPYAQSSQQENFAQQTVVSIYDKVVPGGIGTIQPNWQAIFHQYATLEGYIGNVIIPGGTCTHRLTDSAPVTMSGSKKSRRALGPKPKVSLSPKVKEIEAIPMESSITMTSFDEHGKPNGTYVVHL

SEQ ID NO: 38 - protein, 297 aa, Trichoderma harzianum
NLNKPVITPNFPGNGLASLGQGLMDHLAPTHSTWDDWGAGWIPSDCKSMVQGAGFSPNDIIPFNIHYDDCAQSWTFCRHKNSPLSEVQILDIFGRLPVRMRQFVRHLVFVPGNKSAGSNGDNVLMVGDVDVTVFAHEIGHSLDSHAFDPSYGVPFSTGNVWVSNYNLDSAVPDPYAQSSQQENFAQETVVSLYDKAVPGGIGTIQPNWQAIFHQYATLEGYIGDVIIPGGTCTHRLTDSAPVTMSGSKKSRRALGPKPKVSLSPKVKEIEAIPMESSITMTSFDEHGKPNGTYVVHL

SEQ ID NO: 39 - protein, 297 aa, Trichoderma harzianum
NLNKPVITPNFPGNGLASLGPGLMNNLAPTHSTWDDWGAGWIPSDCKSMVQGAGFSPNDIIPFNIHYDDCAQTWTFCRHKNSPLSEIQILDIFGRLPVRMRQFVRHLVFVPGNKSAGSNGDNVLMVGDVDVTVFAHEIGHSLDSHAFDSSYGVPFSTGNVWVSNYNLDSAVPDPYAQSSQQENFAQQTVVSLYDKVVPGGIGTIQPNWQAIFHQYATLEGYIGNVIIPGGTCTHRLTDSAPVTMSGSKKSRRALGPKPKVSLSPKVKEIEAIPMESSITMTSFDEHGKPNGTYVIHL

SEQ ID NO: 40 - protein, 297 aa, Trichoderma atrobrunneum
NLNKPVITPNFPGNGLASLGPGLMNNLAPTHSTWDDWGAGWIPSDCKSMVQGAGFSPNDIIPFNIHYDDCAQSWTFCRHKNSPLSEIQILDIFGRLPVRMRQFVRHLVFVPGNKSAGSNGDNVLMVGDVDVTVFAHEIGHSLDSHAFDPSYGVPFSTGNVWVSNYNLDSAVPDPYAQSSQQENFAQETVVSLYDKVVPGGIGTIQPNWQAIFHQYATLEGYIGNVIIPGGTCTHRLTDSAPVTMSGSKKSRRALGPKPKVSLSPKVKEIEAIPMESSITMTSFDEHGKPNGTYVVHL

SEQ ID NO: 41 - protein, 297 aa, Trichoderma lentiforme
NLNKPVITPNFPGNGLASLGQGLMDHLAPTHSTWDDWGAGWIPADCKSMVQGAGFSPNDIIPFNIHYDDCAQSWTFCRHKNSPLSEIQILDIFGRLPVRMRQFVRHLVFVPGNKSAGSNGDNVLMVGDVDVTVFAHEIGHSLDSHAFDPSYGVPFSTGNVWVSNYNLDSAVPDPYAQSSQQENFAQETVVSLYDKVVPGGIGTIQPNWQAIFHQYATLEGYIGNVIIPGGTCTHRLTDSPPVTMSGSKKSRRALGPKPKVSLSPKVKEIEAIPMESSITMTSFDEHGRPNGTYVINL

SEQ ID NO: 42 - protein, 297 aa, Trichoderma virens
NLDKPVIQPNFPGNGLASLGQGLMDHLAPTHSTWDDWGAGWIPQDCKSMAQGAGFSANDIIPFNIHYDDCAQSWTFCRHKNSPLSELQILDIFGRLPVRMRSFVRHLVFVPGNKSAGSNGDNVLMVGAVDVTVFAHEIGHSLDSHAFDPSYGVPFSTGSVWTSNYNLDSAVPDPYAQTSQQENFAQETVVSLYDKVVPGGIGTIQPNWKAIFHQYATLQGYIGNVIIPGGTCTHRLTDSPPVTMSSAKLRRGLGPKPNVSLSPKVKEIESIPMESSITMTSFDEHGKPNGTYVVHLK

SEQ ID NO: 43 - protein, 297 aa, Trichoderma asperellum
KLDKPIIQPNFPGGGLASLGQGLLNNLSPTQSTSDNWGAGWIPADCKSLVQGAGLSPFDVTPFNIHYTDCSQTWTFCRHKDSPLSQTDIIDLFGRVPVRMRDFIRHYVFIPGATSAGSSGDNTLMVGNVDITVFLHEIGHSLDSHAFDPSYGAPFSNSNIWISNYNLDSAVTDSYAQTSQQENFAQETVVSVYDKVVPGGIGTIQPNWQAIFHQYATLQGYIGDTIIPGGTCVNRLTDSQPVTMGNSAKFRRGLGPKPTVSLSPDVKEIKPIPMSDSITMTTFDEFGKANGTYVIHL

SEQ ID NO: 44 - protein, 297 aa, Trichoderma gamsii
NLDKPVIQPNFPGGGLASLGQGLLDNLKPTQSTSDVWGAGWIPADCKSLVQGAGLSPNDITPFNIHYTDCSQTWTFCRHKDSPLSQTDIIDLFGRVPVRMRDYIRHYVFIPGNKSAGSSGDNTLMVGDVDITVFLHEIGHSLDSHAFDPSYGVPFSTGNVWISNYNQDSAVTDSYAQSSQQENFAQETVVSVYDKNVPGGIGNIQPNWQAIFHQYATLEGYIGDTIIPGGTCVNRLTDSQPVTMSNSAKFRRTLGPKPTVSLSPDVKEIKPIPMADSIKMTSFDEHGKPNGTYVINL

SEQ ID NO: 45 - protein, 297 aa, Trichoderma hamatum
TLNKPVIQPNFPGGGLASLGPGLLNNLAITSSTSDVWGAGWIPADCKSLVQGAGLSPFDVIPFNIHLSDCSQTWTFCRHKNSPLSQTDIINLFARVPVRMRDFIRHFVFIPGATSAGSNGDNTLMVGNVDITVFLHEIGHSLDSHAFDPSYGVPFSTGNVWVSNYNLDSAVTDSYAQTSQQENFAQETVVSVYDKVVPGGIGTIQPNWQAIFHQYATLEGYIGNTIIPGGTCVNRLTDSQPVPMGNSAKFRRTMGPKPTVSLSPDVKEIKAIPMSDSITMTSFDEFGNVNGTYVIQL

SEQ ID NO: 46 - protein, 293 aa, Niesslia exilis
VLDKPVIQPNFPGGGLDSLSQGLLDNLAPTQSTWDEWGAGWIPQDCKTLAEGSGFSALDITTFNIYYTDCADAWVFCRHKDAPLSEIDMIDLFGRMPVRMRDFVRHIIAVPGNPSAGSSGDNIQMNGGLPITVFVHETGHSLDSHAFSEPFSLSQAWIDAYNQDSAVPDSYAQTSQQENFAQETVVALYDKVVPGGVGTIQPNWNAIFHAYATLQGYIGDVIIPGGTCVNRLTNSAPVPQSNSAKVRIASEKPDVTLRGNVTVLESIPMGSSIELTHFDAEGKPTGRSTVNLG

SEQ ID NO: 47 - protein, 294 aa, Trichoderma brevicompactum
ALNKPVIQPNFPGNGLASLGQGIMQHLTPTQSTWDDWGAGWIPQDCKSIVQGAGFSPNDIIPFNIHYTDCGQTWTFCRHKNSPLSEINIIDIFGRLPVRMRQFVRHLVFVPGNKSAGSNGDNILMVGPVDVTVFIHEIGHSLDSHAFDPSYGVPFSTGNVWVSNYNLDSAVPDPYAQSSQQENFAQHTVVSIFDKVVPGGIGTIQPNWQAIFHQYATLEGYIGDTIIPGGTCTHRLTDSPPVTMASKRRGLGPKPKVALSPEVEEIESIPMKSTFKMTSFDEAGKPSGNYVINL

SEQ ID NO: 48 - protein, 293 aa, Trichoderma pleuroti
NLNKPVIQPNFPSLAPGLMSHLSPTHSTWDDWGAGWIPRDCKSMVQGAGFSPNDIIPFNIHYDDCGQTWTFCRHKKSPLSELQILDMFGRLPVRMRSFIRHMVFVPGTRSAGSNGDNILVVGDGDITLFAHEIGHSLDSHAFDPSYGVPFSTGNVWVSNYNLDSAVSDSYAQSSQQENFAQETVISLFDKVVPGGIGTIQPNWRAIFHQYATLQGYIGNVIIPGGTCTHRLTDSPPVPMSGSAKSRRGLGLKPDVSLTPEVKEIEAIPMESSITMTSFNEQGKPSGTYVIHLK

SEQ ID NO: 49 - protein, 285 aa, Trichoderma brevicompactum
GNPDKPEVSPRIPYDRLESGLRNNLHPTQSTWDYWGAGWIPQGCKDLAKTYHLNPADFTIFNVHYTDCSASWTFCRHKDAGASEIDMIDIFGRLPVRMRSYIRHLMATPGLNDGAAAWTWSSGDCVVESAPNLATLVHEVSHSLDWHALPQYQQPFSSSSIWQDNYNQDSATPTGYGRTNWMENFAETGTVGVYDKVVPGGIGNIEPNWNAIFHQYATYQGYLGDKIIPGGSCANRFEDTPPVAMSNSVKFKITSPKPEIGIDFVGKNITQIIPSKEVEGLYIKG

SEQ ID NO: 50 - protein, 285 aa, Trichoderma hamatum
GNPDKPEVNPHIPYDRLESDLRNNLHPTQSTWDYWGAGWIPQGCKDLANTYHLNPADFTIFNVHYTDCSASWTFCRHKNAGASEIDMIDIFGRLPVHMRSYIRHLVATPGLNDGAAAWTWSSGDCVVESAPNLATLVHEVSHSLDWHALPQYQQPFSSSSIWQDNYNQDSATPTGYGRTNWMENFAETGMVGVYDKVVPGGIGNIEPNWNAIFHQYATYQGYLGDKIIPGGSCANRFDDTPPVAMSNSAKVKVNSPKPGTRIDFAGTNITQIIPSKEVEGLYIKG

SEQ ID NO: 51 - protein, 285 aa, Trichoderma gamsii
GNPDKPEVNPHIPYDRLESDLRNNLHPTQSTWDYWGAGWIPQGCKDLANTYHLNPADFTVFNVHYTDCSASWTFCRHKDAGASEIDMIDMLGRLPVRMRSYTRHLMATPGLNDGAAAWTWSTGDTVVESAPNLATLVHEVSHSLDWHALPQYQQPFSSSSIWQDNYNQDSATPTGYGRTNWMENFAETGMVGVYDKVVPGGIGNIESNWNSIFHQYATYQGYLGDKIIPGGSCANRFDDTAPVTMSNSAKFRVTSPKPETGIDFAGKNITQIIPSKEVEGLFIKG

SEQ ID NO: 52 - protein, 290 aa, Diplodia seriata
DLLRPEIDTVFADGLGPLDQPLVDNLPLTSYTLNQWAPGWIPADCKAMIEGQNMSATEMTVWSVAYADCVDPWVMCQHPSAELNLTAMIDLFGRLPVKMRSLVRHLAAIPGRPWAYNSGTTGRISVFAHEVGHSMDYHGYFNDTRGQFSLSSLWLDNFAKDSGVADYYANTDQGENHSQEVVVALFDANVPGGLGSVTGNWSAILHQYATIQGFVGDGGDTLTPGGACSHRHNNSAAVPMTTEDAAQGPPPDVELTGPVHVYAFKPMEPVANATVVKEDGVAAPMVVKVF

SEQ ID NO: 53 - protein, 336 aa, Trichoderma harzianum
QNLNKPVLFTDGLSPHVDSVFFQHLTPTQSTWDQWGWGWIPQACLSVVQGTQFSPYDIEVYNVHYTDCASAVVMCRHNQAQMSIINMIDAFGRVPIHDRQWTQHILALPASSCSAGTGGAVTTFYGPCYIPSVFVHELTHTLDAYVNDQTGSTGSVHVYSTTSSYLNSVNQDSCVPDGYANTSPQEDYAQTSVLALYEKVNPGGLDPIGNWRCLVNAKNNLDSIIGQELTPGGSCTHRWADSAIVCMGPAAGCASSKRGFSTRRGARVAGPKPEPANKKGDPLVSNLTPDLTSAVNAKIDDFSHLVSNHTASIEATIRINAWNKAAGKKTVPVPTA

SEQ ID NO: 54 - protein, 290 aa, Fusarium tricinctum
QLSKPVMNPPVPFSDLDPTLFKYLKSTPHNRDQWGWGWLPARCKAIADQQGLNAYDIDVYNVHYSDCNQAWVMCRHHLSPLSIDTMAENFGRIPVRMRNFVRVQFALPQKDSSFGGATFSDLGDILWYGDVSKTLRFWIHEAGHTIDRNINPGSNDDYSKSSAWLNEYNKDGFICDDYAKNNAAENFAQEVIVALFDKVVPGGLGSVSPNWNSIFHQYATVQSKLGDILIPGGTCNRKFADDTIVCMGPAANCNSKANAKFRLKADKTQDAAIVAASDNPPIEDLFEVEE

SEQ ID NO: 55 - protein, 279 aa, Aspergillus calidoustus
QLPKPIMSPAVPFDHTDPILYQHLGSTPSTYDQWGWGWLPLRCKAVAEARGLNPYDVNVYNVHYEDCDQAWVMCRHHGAQVSLDQMIDNFGRLPVRLRNIVRHQFAVPGEGLGAYTYSDLGDIVFTGDIGHLLRFWVHEAGHAVDRNINPSQGDYSSSQAWINEYNKDGYICDEYAKNNMAENFAQEVIVALFDKVVPGGIGTIVPNWNDIFHQYATVQAVMGDMLIPGGFCNRHFADDTIVCMGPAAGCENSKRDYEGLNSTETYTAEAEEPTVCTLA

SEQ ID NO: 56 - protein, 290 aa, Xylariomycetidae sp.
DDTPPFKPVISPAFDFYGTLEDGLRANLAPTQSTWDQWGEGWIPDDCKWYAEYYGYAANEFEVFNVKYTDCDEAWIMCRHKQATVSQTGMIDLFGRLPLGMREYVKHVVMAPETKANTVASSYRGTIAFDQDYFSIYNLVHETGHCMDGTAALDVFPDGISSSSIWQDNYNQDSAVVRDYARSSWVENFAETGTVAVYDKVVPGGIGPIQPNWEQIYHQYATYNGYLSDIITPGAKDKCTQRRENSETVAMGASSRIMRSSPPDTSFKSDVKVMPAGANDEFFFEHSRDH

SEQ ID NO: 57 - protein, 285 aa, Peziza echinospora
QLDKPLVVPAMPALDAGLLKHLAAPAYTTSQWEWGWIPQWCKDEAVSEKLSPYDIEVFNIHYNDCKDVWIFCRHNAAQASQKDMIDIFGRMPVRTRQFVRHILAVPGTISAGSSGDNIALRGTGPNLISVLIHEAAHSMDSHAVDSLSKPFHNTAEWLAAYKADSAISDDYAQTSQSENFAQTVVIAVFDKVVPGGIGSVQPSWAAIKNQYTTAQKWLGDLIIPGGTCTARLENSPVASMGRASGNGARMRRRARGGKPDVSLSKDTKVIPAIAGKKIHITSFAQ

SEQ ID NO: 58 - protein, 282 aa, Talaromyces proteolyticus
QTLDKPALTPDLDYLEQGAIDNLAPTQSTNDQWGSGWIPSDCKDIANNEGKNPDDFEIYNVHYDDCDDAWVFCRHKDTPTDLITTIDIFGRLPVAMRSWVRHVLTIPGDNYAYNSNGNIAFFGTTSGIMDVAIHETGHSLDLLGAYGDQLSSSQDWLDNYNQDSNVPDNYAQTNQIENVEQNTVVSVYDKVVPGGFGSAQPNWNNIFHQYATLQGKAGDQIIPGGSCDRHLVNSETVSTSDSAAAAMVDAVDKPVTSFKGNYTKILKTHAKFNTKESCYFGN

SEQ ID NO: 59 - protein, 286 aa, Aspergillus tanneri
KRLDKPALTPDLGYLLQGSVDNLHPTHSTWDKWGDGWIPEDCKSMAQNPNLNLNLNPSDFEVYNVHYDDCGDAWVLCRHKDSPVDLVTTVDIFGRLPVRMRDWVRHVMTVPGNDYAFNLNGNIVFSGRTSTNMDVAIHETGHSLDLLKAYGEVLSSSQTWIDNYNQDSNVPDNYAQSSQIENVAQNTVVSVFDKIVPGGFGSVQPNWHNIFHQYATLKSKAGDLILPGGQCDRRLKDSSPIPMDSNANEQGLLSWKPRTSFVNTYNNIVTNFEPFNTAEHCKESFS

SEQ ID NO: 60 - protein, 325 aa, Pseudogymnoascus sp. WSF 3629
MASSLMKAAAAAVLFGAPALAGLGKPTLFSDGLSPHVDSVFWEHLTPTQSTWDRWGWGWIPQTCFNHANDNNVSPYDMEIYNVHYTDCGSAFVFCRHNAANLGIIDMIDLFGRLPIHERQYITNVIAVPGGGSAYETSAIVVFQGPVGTPSVFQHEVGHAVDAYHNSVGSSETSLFKDAINADSCVPDDYSNSSNAEDYTQVSVLALYEIVNPGGLDPIGNWRCLERQKNAVNQLQGDAMIPGGTCTFRWADAAIVSMGPATGNGKRAVGAKPTPNLVAGQPGVKELPFEHSSKVTQYKDLHFNEAESAKASNFAVERRQVAFRG

SEQ ID NO: 61 - protein, 325 aa, Pseudogymnoascus sp. 23342-1-I1
MVSSLIKAAAAAVLFGSPALAELGKPTLFSDGLSPHVDSVFWEHLKPTQSTYDKWAWGWIPETCFNHANDNNVSPYDMEMYNVHYTDCDSAFVFCRHNAANLAIAEMIDLFGRLPIHERQYITNVIAVPGAGSAYETSAIVVFQGPVGTPSVFQHEVGHAVDAYHHDAQSSETDIFKNAINADTCVPDDYSNSSNTEDYTQISVLALYEIVNPGGLDPIGNWRCLQNQKNVVTRLQGDAMTPGGTCTFRWADAAIVSMGPATGNGKRAVGAKPTPNLVAGQPGVKVLPFEHSSKVTKYKNLQFNEAESAKASKFAVERRRVKFRA

SEQ ID NO: 62 - protein, 369 aa, Carpinus fangiana
MAYLQRYNQPLLALMLLHCLAWGPGPTSGGSPGTHCRSGSLKVKLTLCILLSFLPKNSPLFVTMMTQIKSIAILAFAAAAMAQKLDKPALTPDLDYLNQGNIDNHGPTQSTTAGYEAGQIPADCKDRAQAEGHDPADMEVTYVTYTDCADPWVFCRHKDSVPSLVDLQDMFGRVPVGMRDYVKHVMSFPGGQGAYNYGGDIVFFDTAGLTFDVWLHESAHSLDLQGAITGDSATTYSSTDNWVNNYNQDPNVADNYAQSNMVENFAQETVVAIYDKAVPGGFGGVQPSYNNLFHQYATVQGDAGAKIITGGTCNRALQPSGPVSISSTARRTTSAYERPEHTFSQKYSNLRAPNLKANTKDTCKIGHKH

SEQ ID NO: 63 - protein, 338 aa, Pseudogymnoascus sp. 05NY08
MISSAMKAIIAALALLSAPVLADLGKPVLFKDGLGPHVNTKLMAHMPSTKSTHKEWAHGTIPESCATQGKNANPPLKPSDFHIFDVHYTDCAEAWIMCRHKDAQLSINQLIDYFGRLPVHERQSVSYVMGVPGGGSAYEIGGMVVFQGNTASISVFQHEVGHAVDAYNNADGTSSSKPIFAEALKKDSCVPDAYANTNKVEDWTQVSVLLMYDKVVPGGLNTIGANWACLGAQLVAVANRQGAAMNLGGKCTKRLPYGKVISMGGATARGESAVDIPVMTLVPGHPLVVDVDTNRGGEVTHFTDLTFNETEDALAAKRQAAWNAEAAKSTGRRVRRNF

SEQ ID NO: 64 - protein, 336 aa, Pseudogymnoascus sp. WSF 3629
MFSSVVKAAVAAIALFGAPALAGLGKPTLFKDGLSPHVNAEFMQYMPSTPSTSGAWSWGWIPKRCADEANRPKVKFSWYDFEVFNVQYEDCEEAWIFCRHTKAELSQSQLIDYFGRLPVHERQSVSYVMAVPGGGSAYEIGGMVVFQGNTASVSVFQHEVGHAVDAYNNADGSSSSTKEFLDAIDGDTCVPDDYANSSNVEDYTQVGVLLMYNTVVPGGLDKIGADYSCLSKQLAVVAARQQAAMELGGTCTWRQTYGDVISMGPASGNNKRSIGPKPVMSLVSGHPLAVEKDINRGGEVTVYDNIISNEAEDAKAAEKQAAWNAVARHRAVRSNI

SEQ ID NO: 65 - protein, 337 aa, Pseudogymnoascus sp. VKM F-3808
MFSSIMKAAAVASFLFSAPALAANLGKPTLFKDGLSPHVNDKFIQYMPSTPSTNGAWSWGWIPQRCADEANRADPPLSLYDFEVFNVQYEDCEEAWIFCRHTQAQLTQSQLIDYFGRLPVHERQSVSYVIGVPGGGSAYEIGGLVVFQGATASVSVFQHEVGHAVDAYNNADGTSSSTKEFLDAIDGDTCVPDDYANSSNVEDYTQVGVLLMYNTVVPGGLDTIGTDYSCLSKQLAVVAARQQAAMELGGTCTWRQTYGEVISMGPASGNNKRAIGPKPIMNLVPGHPLAIEKDINRGGAVTHYGEVESNKTADALAAQKQKEWNAVAKHRAVRRSI

SEQ ID NO: 66 - protein, 291 aa, Dactylella cylindrospora
MVQFTRSILFAALSVSGAFAQLKTLLDYPDGLSGPFNPGFLERTAAPLERIERWGEGWIPQGCKDRFLSKNLLASDAEVFNVKYSDCDRAWTFCRHNTAELSIEQMADSFGRMPVHMRSLVRHAIALPQNGCSAQAFTDRGDIVMFGPCTGVTVWLHETGHQLDSRLKPAERFSGTQEWLEALGADTCVPDSYANTNTVEDFAQVTVVSQYNTLLGFKPTEKYPGCLDHRHGLLEHNFRALYLEYGGSCSSRPADSEIVSTATTKREVMRLMRRANETDPASIGPCFFDEM

SEQ ID NO: 67 - protein, 325 aa, Pseudovirgaria hyperparasitica
MHATSVSSAVFLALIAIVDANQGSFAPLTKRELDKPVIQPPFYDNKLVPALNESLQTTEHTIDLWANNSIPSICKSAIEEQAVDGVLLTSGPVEVFSVTYADCDTPWIMCRHPTASASRQTMAELFGKLPVRHRSFVRHLSAAGGDSGAYSSGDNVFMAGDSSLAVTVYGHEVGHSLDAHFFGNETGFTETDAWLEAYDEDEVVVQDYAQASQAENFAQMVNVAIYTLNFPGGIANLAPNWQQFSHMLDLMTNELKDVLTQGGECRNRLENSPLVSKSGFGSQTSSVLSSESSSTVMTIASPTSSPAPGTRTRALHRRQRQHQAK

SEQ ID NO: 68 - protein, 294 aa, Orbilia oligospora
MINLFSNIVAVVAVSAVGVFADLKNKTDYPDGLSGPFTPGLNNYTEAPLEWFTPWGAGWIPKGCKDRFIGKGYDADDAEVFNIKYTDCDQAWTFCRHKDAQLSLEDMADSFGRMSVHMRSLVRHPIALPASSGCSALAYTDIGDIVMFGNCKGLTVWLHETGHQLDARLKPSNRFSGTTPWLEALERDTCVPDSYANTNTVEDFAQVVVVAQHETLLGYKPEEAFPGCLDQRQGLLERTFQSDYLEYVGRCSNRPRDSEIVSTKTTKREKMKMMKRAMAQEGNDPNVVGPCSFP

SEQ ID NO: 69 - protein, 296 aa, Arthrobotrys entomopaga
MTYIANTLIAVLAAAGSTYAQLPEPYVDYPDGLSPVFDQGFFAGTASPSVSIHQWPSGTAPQSCVNELAWNNCISGRAVVYNVTYADCPTPWVFCRCDNADLSITQMADFFGRMPVHLRSVVRHPMAVHQDGCSAYATIGTYDGDIVMQGNCNTQSVWLHETGHQLDARGNGPNTYFSSTSAWTDALFADTCGADDYGNSDILEEFAQMTVISQSDVLFGVVPQQAYPGCLDNQLAALEGAFKVSRLTYGGTCVDRVPDSPTVTAVWKRGVSRRGALDVERVGPCKFNNLTAGHKH

SEQ ID NO: 70 - protein, 341 aa, Pyricularia grisea
MVQVKLAALLAVAGLAAHVSAQDKLNKPPLLPNLDMLWEGLEKKLTTPKTEIKQWKDNIISEDCKTMAEREGVSPNSMIQYEVTYGDCKMPWLLCAAKDHPSNMKDIAKFFGMVPAKLRQATKHAAFFKPKTAKSTAAAYALGDQLAFMMDPKGIGIFLHEVSHTADLGAKDSHFGKVEGRFTDNKIWTDAIQKDQAVSDNYGQNSKHENLAQYAGLVLYDILVDGGLKKIVKDVDKIKNQIDAFKTATRECRWGGNMLKLDQTCTKRPPNAKPIDLGAKPEDKKDDKKDDKKDDKEGKGKGKRKLRFRRDARSMFWPRASGSADSVEKRDLPTNCGLPSA

SEQ ID NO: 71 - protein, 329 aa, Sodiomyces alkalinus F11
MLPIELVQVASLLVLAGVSLAQVPPGHAALFPEGLSVLNTRPRSIGLNNMQGVPFGFTQWAFGTVPQVCYNAAVDDGYCNVYDLEVYDVEYDDCAVPWTFCRCSDSPLSIEAFAGRVGRLPVAARQYARFFSSYGGNSSAFSFRNDITFFGPIEGQSVYIHELSHNLDRLYSPPDSLTSFSGTDEYANAVAADSCSPDDYAKTSYAEHYAQTSVMIAYDVTVGPIDSVRNTSCMANAFNAARKHLAPIWQFNEVGTCTSRLSEDDSPRVCMGPEAPCPGDPVRVASSDSESDPEGFRWAPVVESVDDEELERKRRAEREAGMRGYAEIA

SEQ ID NO: 72 - protein, 340 aa, Neurospora crassa OR74A
MPQAHFFALLLAAVVPAVLADGPPESMGEKFSGLNVLDGNGGLQSLTPTPYTISQWPWGTVPKLCYDTSVNNKYCNPYDLEVYDVRYTDCPIPTTVCRCKNSPMAIDTIAQRVGQLPVKARQYNGYVSSFAGDMCSAYSDSFNNYFFGDCGNSESVFFHELSHNLDRHVAGASINDWYSLSQDWKDTVAKDTCVADHYSKASWLEAYAQVGVMAGYDATVQSIYTQNVGCMVNQVKKVVGQLNSVWRKQPGQMCDRYWIKDTTVCMGPDAEASGHCQASKADVAAESGGVNPVLPDGQQKKHDALVKELQRHAEAAAGISSGKPAADRKTKGKKGTKFRV

SEQ ID NO: 73 - protein, 554 aa, Blastomyces dermatitidis ER-3
MVVLSLLCPLMLLGLASAQIIKDPLVKNVLDFDSTFDASLPAPQKYTYKKWTTYEIEQGLPPDSTWTASYYEKESRHYCRADFSVYNVTYADCPEPWVIGHCAKAEISREATFDLLGRLPSSARGVISDLLNANIERGLSFRWHNKHSVMLGGYFRPADGLKAALAALWVGGPGVPYETFLNAVNADTCVADTRAAESLKRDGSLGDAVEKGFAVAAYLKLVKGTPPFDASCMSNQLKVLGAILDQRWDAPGQCPDKKAPELVKYRSVLFSAGIEVLNTDPVPGSRPAEVVYWPKFKGYPEFCWNEARAQRSNDDLRPRCEPRRLNVFNVTYEDCPDQDPWVICHCSDAQQSLDDVIRRVGQLPPGLRSHMIHLIAFEHSSVGGAAVLPWNMVMIFGEAQDSVYMHEASHCIDRGFYASETFRTAKAKDSCWPTHYSKSADIELFAEIGVLYLYDKSGKTLIQRGHDPACLANGLKAIDAYVGLDYQRGGSKCFKRKPNSKVVYPPGFQIQSPEPYISNAVIENFSNPGDVSSLSSLSSPASPWGDYPREIHGQ

SEQ ID NO: 74 - protein, 310 aa, Karstenula rhodostoma CBS 690.94
MVRFISAAGVALALATSTRAHTVPLSHGMSQLSQRDLAKTALLDNFDALTADPSRIPPIAKGTATKWDAGRYPQNCYDKAREAISDEDARPKCELRDLEIYDVTYEDCPSGRDPWVLCRCSNAELSLQDTIDGMGTVPPGARSNVLHIVTMNGFGGGGGYSDGNSIFCGGAPSKTWFSHESMHCNDRDGFAFSNGDVFNNAINADSCVPDSYANNNPTEDFAQVGTYLNYDINGHPIDYTGKDASCMGNQLNAARDYLAERLDKNGACFNAPGTDGIVNEKRSTTIIEKDPNSSYEKLLPHTPYAHAEFF

SEQ ID NO: 75 - protein, 269 aa, Peniophora sp. CONT
MISVHSIVLALAGAAPALADLGKATLFPNGLHDPLIGLDNLPFQTFTYSGVAVPDLCISHATDAGCDTSTVEAYQANYGDCSEPWTLCRCANAQMSAATMLTRFAQVPPGVRSYVGSALAVSASSCSAGSGGDFIVFNGDCSQSVFDHESGHSLDQNTSPSSEWTNALAASSCVPDAYANTSPAEDFAQVNVCYIYQSRVGALPADASCMQAQLAVFDNDARIHGAQTATTCNAGVRPFQLSDNKVTSKNLTNALYNGKIETKFFHANP

SEQ ID NO: 76 - protein, 319 aa, Exidia glandulosa HHB12029
MLQFVSLAVVLASSPLALAALGKDTLFKNGLTDPLADYYDSIPKPPSSRVDMPAPDMCIQHAEGKCDASAVEAVSVKYDDCDVPWTLCRCNDANMSMDTIVDRFGQLPPGIRSYVGSLIAVHADGPSAGSSGDFITFNGDCNTSVFLHEASHSLDQGTSPSQGWTDALGASSCVPDDYANSSPAEDFAQVDVLYVYTKRHGPLPADPSCLQAQLDYMANTPRIRDAEASATCNADVRPFTLDPPAAPAPEPAPEPTPTPAPEPAPEPTPTEAPAPPPETPAPTPTPVPAPAPSQCPAGMRRRSEKEARRLARRLAPHRR

SEQ ID NO: 77 - protein, 346 aa, Ascodesmis nigricans
MRFELSTSSLSIALTALFLSTTATAQTNSRYPKGLYSEGLKVLDPPNLASSAVSATSLRAIPSEIPYSCYGTALSRENKELPAINCAISSLEVFEVTYPDCERSWKMCRCSDADLSREDMMELFGMLSPNLRAQVRNLMAFKTSKSAHAYTSNEDVVVFGDLRHWTVITHEAAHVLDSRKGGLSETAVYEDAIKKDTCTADYYAANAMTRGDVKEPWAQTFVVNTFRKLGGVLKADAGCMQNQLDVVADQTDDFVKKSRCDMSMATTEGPMFERSPSEVLDKSITIENFPASLTAVSPSKTAYPSSDIVIRMKESEDEKSAGTTALRTTGWGIATSILMLQLWYMN

SEQ ID NO: 78 - protein, 583 aa, Planoprotostelium fungivorum
MRTTLFVVAMIAAVLAMDLSGKRPLWDGMKDYPLPSAAYKLQPAADSGNCKVEVCNDGKAISNTWVIYEDGPRILLCVCSNAFMNVSGMAYRQGQVPIAVRSSVATMTAGSNGKGGGAWTMFNDIAYFGDFVANVFLHESGHALDFAQGNMYGSMGLSGADQWHSAIDSSSCVPDPYAKTNEVEDWAQNTVLRYWAKVLGKAWDNDETSCMKNQLKYSMEALPERPAFDVKSSFRISPVNNKNLVLATLNDSTSDNSPVALVKISDRASQMWKIMPTAYDWHILCEGNSYKCLDNQRDNNVGKQAIITYRTAFISMQHRFISVGPNTYKIIQRTSGLALSVGCNGRPGDRPSYVRDGDDSCLKWTITNSKSSCDNSVGSELSSDATSTLYSDQQVLTSPDCNLRLVMQPDGNAVIYERNNRVRWASNSFCSGPNKLVMQPDGNLVVYDKNMGAYWSTGTYNRGVGPYKLNMDNNAKLTLRDSTGSVFTYSLFNDVYKRKTVRSSMKLSWCDLGKTENRYSKAQSFPGINSTNVGEVMAKVHNLAELSTLSLDKMVKLLGKKNGEALHHFMNHKGRVEFENAVE

SEQ ID NO: 79 - protein, 490 aa, Planoprotostelium fungivorum
MRGFLSLALIALAVGFDLSNKDYIWGDGLSDHISLAAVTPKPSGFTVHPSDLSSCSGWDSCDTDGQSPYYPNDGLPVNSTWLINFDDGTWTELCICEGYVMDENEMAKTLSYVPPSVRSSVNRVTQKPTGNGAVTLGNSAIYFGRYNVEVFVHESSHSFDWSNEISGKQEWLDAIGNDTCVPDPYASSNEAEDWAQTSVLYWFLTYSNANNETLHNATFECWSRSREVAASYMPNNTSREFDPTHRVTIMPKTSSGSLNYLDNGEVSIDSPHSSGWQLVTASYNYYIICESGDFTLSCLDALGQNQENDPIYMYYPRNGGVNQQWSLLPDGEGYYKIINRANGMALTTYGCGTGNATASVVFGADGDQDCNKWKIIDPNASTTSQDDTTTTEVDTSTTSQVTSSTTSQATSSTTSQITSSTTSQVASSTTSDTSATVVDTQSSTDSSTSEVTSVSQPDTSAIVSTVPASQASHLHLAVGSLFCLVIMAAF

(19) United States
(12) Patent Application Publication
(10) Pub. No.: US 2025/0051821 A1
(43) Pub. Date: Feb. 13, 2025

(54) COMPOSITIONS AND METHODS INVOLVING PROTEASES SPECIFIC FOR MANNOSE-MODIFIED PROTEINS

(71) Applicant: DANISCO US INC., PALO ALTO, CA (US)

(72) Inventors: BRADLEY R. KELEMEN, MENLO PARK, CA (US); MARINA CHOW, SAN FRANCISCO, CA (US); KIRSTIN Y NOSE CROTTY, SAN JOSE, CA (US); THOMAS P GRAYCAR, PACIFICA, CA (US); JEFFREY VEACH MILLER, MENLO PARK, CA (US); ROMAN RABINOVICH, LATHROP, CA (US); STEVEN CARY ROTHMAN, SUNNYVALE, CA (US); TREVOR STARR, SAN JOSE, CA (US); ZHONGMEI TANG, SHANGHAI (CN); AMR R. TOPPOZADA, SAN CARLOS, CA (US); MONICA LESLY TSE, CUPPERTINO, CA (US); JULIA YAGER, WILMINGTON, DE (US); ZHENGHONG ZHANG, SHANGHAI (CN)

(21) Appl. No.: 18/719,904

(22) PCT Filed: Dec. 15, 2022
(86) PCT No.: PCT/US2022/081696

§ 371 Date: Jun. 14, 2024

Related U.S. Application Data
(60) Provisional application No. 63/289,652, filed on Dec. 15, 2021.

(51) Int. Cl. C12P 21/00; C12N 9/64; C12P 21/06

(52) U.S. Cl. CPC C12P 21/005; C12N 9/6416; C12P 21/06; C12Y 304/24057

(57) ABSTRACT

Disclosed are compositions and methods involving proteases specific for mannose-modified proteins. The compositions and methods are particularly useful for making linker-specific cleavages in proteins produced by yeast and fungal cells. One use of the compositions and methods is for agglomerating yeast and yeast components in fermentation products. Another use of the composition is for producing a fraction of protein with reduced carbohydrate content.

### CROSS REFERENCE TO RELATED APPLICATIONS

This application claims the benefit of U.S. Application No. 63/289,652, filed Dec. 15, 2021, which is hereby incorporated by reference in its entirety,

### TECHNICAL FIELD

Disclosed are compositions and methods involving proteases specific for mannose-modified proteins. The compositions and methods are particularly useful for making linker-specific cleavages in proteins produced by yeast and fungal cells. One use of the compositions and methods is for agglomerating yeast and yeast components in fermentation products. Another use of the composition is for producing a fraction of protein with reduced carbohydrate content.

### BACKGROUND

Protein glycosylation is a common natural modification of polypeptide chains. Glycosylation refers to the attachment of carbohydrates to functional groups of proteins to modulate folding, stability, solubility and protein-protein interactions. Different types of protein glycosylation are known, which can generally be categorized as N-linked glycosylation, in which carbohydrates are attached to amino groups of an asparagine or arginine residues, and O-linked glycosylation, in which carbohydrates are attached to hydroxyl groups of serine, threonine or tyrosine residues.

In a particular form of O-linked glycosylation, mannose sugars are directly attached to serine and threonine residues, often in linker domains of modular proteins. Such modifications occur predominantly in yeast and fungal cells but infrequently in bacterial cells. While this form of O-linked glycosylation has previously been described, the ability to exploit it for commercial purposes has not.

### SUMMARY

The present compositions and methods involve proteases specific for mannose-modified proteins. Aspects and embodiments of the compositions and methods are summarized in the following separately-numbered paragraphs:

1. In one aspect, a method for modifying a mannose-decorated amino acid sequence present in a target protein is provided, comprising contacting the protein with a recombinant polypeptide having mannose-specific glycoprotease activity, wherein the contacting occurs in a non-naturally occurring environment.

2. In some embodiments of the method of paragraph 1, the modification is proteolysis.

3. In some embodiments of the method of paragraph 1 or 2, the mannose-decorated amino acid sequence is in the linker region of a target protein.

4. In some embodiments of the method of any of the preceding paragraphs, the target protein is present on a hydrophobic surface of yeast or fungal cells, cell bodies or cellular components.

5. In some embodiments of the method of paragraph 4, the contacting results in aggregation of the cells, cell bodies or cellular components.

6. In some embodiments of the method of any of the preceding paragraphs, the contacting occurs in an industrial or pharmaceutical reaction vessel.

7. In some embodiments of the method of any of the preceding paragraphs, the target protein and recombinant polypeptide having mannose-specific glycoprotease activity are from different organisms.

8. In another aspect, a method for agglomerating organisms displaying mannose-decorated amino acid sequences on their surface is provided, comprising contacting the organisms with a recombinant polypeptide having mannose-specific glycoprotease activity.

9. In some embodiments of the method of paragraph 8, the organism is yeast or fungi.

10. In some embodiments of the method of paragraph 9, the organism is a Saccharomyces sp.

11. In another aspect, a method for modifying a fermentation product produced by yeasts cells and comprising yeast cells, cell bodies and/or cell components is provided, comprising contacting the fermentation product with a recombinant polypeptide having mannose-specific glycoprotease activity to produce a modified fermentation product having dissolved solids with reduced optical density and/or an insoluble fraction enriched for protein.

12. In some embodiments of the method of paragraph 11, the reduced optical density results from aggregation of the yeast cells, cell bodies and/or cell components.

13. In some embodiments of the method of paragraph 11 or 12, the fermentation product is stillage from an ethanol fermentation process.

14. In some embodiments of the method of any of the preceding paragraphs, the recombinant polypeptide having mannose-specific glycoprotease activity complies with the Hidden Markov Model TreSub-21374_NRBlast_HSS-id35-qc70_T2k.

15. In some embodiments of the method of any of the preceding paragraphs, the recombinant polypeptide having mannose-specific glycoprotease activity has at least 90% amino acid sequence identity to an amino acid sequence selected from the group consisting of SEQ ID NO: 1 (IFF05497), SEQ ID NO: 2 (IFF21332), SEQ ID NO: 3 (IFF21333), SEQ ID NO: 4 (IFF21334), SEQ ID NO: 5 (IFF21335), SEQ ID NO: 6 (IFF21338), SEQ ID NO: 7 (IFF21340), SEQ ID NO: 8 (IFF21347), SEQ ID NO: 9 (IFF21350), SEQ ID NO: 11 (IFF21354), SEQ ID NO: 12 (IFF21359), SEQ ID NO: 13 (IFF21360), SEQ ID NO: 15 (IFF21363), SEQ ID NO: 16 (IFF21364), SEQ ID NO: 17 (IFF21365), SEQ ID NO: 18 (IFF21372), SEQ ID NO: 19 (IFF21374), SEQ ID NO: 20 (IFF21375), SEQ ID NO: 21 (IFF21378), SEQ ID NO: 22 (IFF21379), SEQ ID NO: 23 (IFF21380), SEQ ID NO: 24 (IFF21344), SEQ ID NO: 26 (IFF21366), SEQ ID NO: 36 (IFF21331), SEQ ID NO: 37 (IFF21336), SEQ ID NO: 38 (IFF21337), SEQ ID NO: 39 (IFF21339), SEQ ID NO: 40 (IFF21341), SEQ ID NO: 41 IFF21342), SEQ ID NO: 42 (IFF21343), SEQ ID NO: 43 (IFF21345), SEQ ID NO: 44 (IFF21346), SEQ ID NO: 45 (IFF21348), SEQ ID NO: 46 (IFF21349), SEQ ID NO: 47 (IFF21351), SEQ ID NO: 48 (IFF21352), SEQ ID NO: 53 (IFF21367), SEQ ID NO: 54 (IFF21368), SEQ ID NO: 55 (IFF21369), SEQ ID NO: 57 (IFF21371), and SEQ ID NO: 59 (IFF21377).

15. In another aspect, a stillage product obtained by the method of paragraph 13 is provided.

16. In anoyther aspect, a recombinant polypeptide having mannose-specific glycoprotease activity and: (a) having at least 90% amino acid sequence identity to an amino acid sequence selected from the group consisting of SEQ ID NO: 1 (IFF05497), SEQ ID NO: 2 (IFF21332), SEQ ID NO: 3 (IFF21333), SEQ ID NO: 4 (IFF21334), SEQ ID NO: 5 (IFF21335), SEQ ID NO: 6 (IFF21338), SEQ ID NO: 7 (IFF21340), SEQ ID NO: 8 (IFF21347), SEQ ID NO: 9 (IFF21350), SEQ ID NO: 11 (IFF21354), SEQ ID NO: 12 (IFF21359), SEQ ID NO: 13 (IFF21360), SEQ ID NO: 15 (IFF21363), SEQ ID NO: 16 (IFF21364), SEQ ID NO: 17 (IFF21365), SEQ ID NO: 18 (IFF21372), SEQ ID NO: 19 (IFF21374), SEQ ID NO: 20 (IFF21375), SEQ ID NO: 21 (IFF21378), SEQ ID NO: 22 (IFF21379), SEQ ID NO: 23 (IFF21380), SEQ ID NO: 24 (IFF21344), SEQ ID NO: 26 (IFF21366), SEQ ID NO: 36 (IFF21331), SEQ ID NO: 37 (IFF21336), SEQ ID NO: 38 (IFF21337), SEQ ID NO: 39 (IFF21339), SEQ ID NO: 40 (IFF21341), SEQ ID NO: 41 IFF21342), SEQ ID NO: 42 (IFF21343), SEQ ID NO: 43 (IFF21345), SEQ ID NO: 44 (IFF21346), SEQ ID NO: 45 (IFF21348), SEQ ID NO: 46 (IFF21349), SEQ ID NO: 47 (IFF21351), SEQ ID NO: 48 (IFF21352), SEQ ID NO: 53 (IFF21367), SEQ ID NO: 54 (IFF21368), SEQ ID NO: 55 (IFF21369), SEQ ID NO: 57 (IFF21371), and SEQ ID NO: 59 (IFF21377), and/or (b) being identifiable by the Hidden Markov Model TreSub-21374_NRBlast_HSS-id35-qc70_T2k, is provided.

These and other aspects and embodiments of the compositions and methods will be apparent from the present description and drawings.

### BRIEF DESCRIPTION OF THE DRAWINGS

FIG. 1 shows images of whole stillage samples in conical tubes that were either untreated (A) or treated with IFF05497 (B).

FIG. 2 is a bar graph showing the levels of total suspended solids in whole stillage supernatants following treatment with (B) or without IFF05497 (A).

FIG. 3 is a line graph showing that the diffusion coefficient of size-selected particles in thin stillage changes with time as a result of the addition of IFF05497 at a dilution of 6 nm, (circles), 3 nm, (triangles) and 1:5 nm, (squares). Water (+) was used as a control.

FIG. 4 is a line graph showing that the diffusion coefficient of size-selected particles in thin stillage decreases more rapidly as a result of increasing addition of IFF05497 (Enzyme).

FIG. 5 is a bar graph showing the amount of solids recovered from thin stillage following treatment with (B) or without IFF05497 (A).

FIG. 6 is a bar graph showing the amount of total suspended solids in thin stillage supernatant following treatment with (B) or without IFF05497 (A).

FIG. 7 includes two light microscope images showing untreated yeast (A) and yeast treated with purified IFF05497 (B).

FIG. 8 includes two light microscope images showing untreated inactivated yeast (A) and inactivated yeast treated with purified IFF05497 (B).

FIG. 9 is a series of images of SDS-PAGE gels loaded with protein samples treated with IFF05497 and showing a gel mobility shifts. Lane designations are shown in Table 10.

FIG. 10 is an image of an SDS-PAGE gel loaded with protein samples including IFF01073 produced in T. reesei or E. coli and incubated with or without IFF05497. Lane designations are shown in Table 12.

FIG. 11 is an image of an SDS-PAGE gel loaded with protein samples treated with IFF05497 or related proteins. Lane designations are shown in Table 12.

FIG. 12 is a graph showing a reverse phase chromatogram of IFF05588 (solid line) and IFF05588 treated with IFF21374 (dotted line).

### INCORPORATION OF ELECTRONIC SUBMISSIONS

Sequence listing 20221212_NB41708_ST26SequenceListing accompanies the present application under 37 CFR 1.821.

### REFERENCE TO SEQUENCE LISTING SUBMITTED ELECTRONICALLY

The official copy of the sequence listing is submitted electronically as an XML formatted sequence listing with a file named 20221212_NB41708_ST26SequenceListing created on Dec. 5, 2022 and having a size of 92,968 bytes and is filed concurrently with the specification. The sequence listing contained in this XML formatted document is part of the specification and is herein incorporated by reference in its entirety.

Hidden Markov Model TreSub-21374_NRBlast_HSS-id35-qc70_T2k accompanies this application and is submitted electronically under 37 C.F.R. 1.96.

### DETAILED DESCRIPTION

### 1. Definitions and Abbreviations

Prior to describing the various aspects and embodiments of the present compositions and methods, the following definitions and abbreviations are described.

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

The present document is organized into a number of sections for case of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art. The following terms are defined, below, for clarity.

As used herein, a “mannose-decorated” amino acid sequence is a contiguous amino acid sequence having a form of O-linked glycosylation in which mannose sugars are directly attached to serine and threonine residues.

As used herein, a “target” protein is a preselected or potential protein of interested having mannose-decorated amino acid sequence.

As used herein, “mannose-specific glycoprotease activity” refers to proteolytic activity with respect to contiguous amino acid sequences having a form of O-linked glycosylation in which mannose sugars are directly attached to serine and threonine residues.

As used herein, “a linker” is contiguous amino acid sequences separating distinct domains in a modular protein, such as a core region and binding domain.

As used here, the term “contacting” refers to bringing a plurality of components into physical proximity, e.g., to facility a chemical reaction.

As used herein, a “recombinant polypeptide” is a polypeptide made in a heterologous organism or a polypeptide expressed from a human-manipulated gene.

As used here, “agglomerating” refers to forming a single mass from a plurality of smaller masses.

As used herein, “disrupting an organism” refers to lysing or breaking open intact cells.

As used herein, “whole stillage” is the byproduct of a dry-grind ethanol production facility following distillation.

As used herein, “thin stillage” is the liquid portion of whole stillage following separation of solid materials.

As used herein, “distillers' grains (DG)” is the solid/slurry component of whole stillage. As used herein, “distillers' dried grains (DDG) is DG that have been dried.

As used herein, “distillers' dried grains with solutes (DDGS) is DG that has been dried along with the concentrated thin stillage for added nutritional value.

As used herein, The term “about” refers to +15% to the referenced value.

The following abbreviations/acronyms have the following meanings unless otherwise specified:

| | |
|---|---|
| EC | Enzyme Commission |
| ° C. | degrees Centigrade |
| g or gm | grams |
| μg | micrograms |
| mg | milligrams |
| kg | kilograms |
| μL and μl | microliters |
| mL and ml | milliliters |
| nm | nanometer |
| U | units |
| min | minute |
| rpm | revolutions per minute |
| hr | hour |
| CAZy | Carbohydrate-Active Enzymes database |
| DG | distillers' grains |
| DDG | distillers' dried grains |
| DDGS | distillers' dried grains with solutes |
| rcf | relative centrifugal force |
| sd | standard deviation |
| HMM | Hidden Markov Model (HMM) |
| RI | refractive index |

### 2. Proteases Specific for Mannose-Modified Proteins

In a particular form of O-linked glycosylation, mannose sugars are directly attached to serine and threonine residues. While this form of glycosylation has been described, the prevalence of this form of glycosylation in the linker regions of certain proteins, and the ability to selectively proteolize the linker regions of such proteins using a specific class of protease, has heretofore neither been described nor exploited. O-linked mannose glycosylation occur predominantly in yeast and fungal cells but infrequently in plant cells and in bacterial cells. Most notably, O-linked mannose glycosylation occur in proteins expressed and modified by yeast, the importance of which to brewing, wine making, pharmacology and industry cannot be overstated. While direct mannose linkages have been reported to improve the resistance of peptides (and by extension, proteins) to proteolysis, such linkages have not been targeted as a means of controlling protein activity or physical properties.

Applicants initially identified recombinant polypeptides that demonstrated unexpected properties when incubated in the presence of various forms of stillage from grain ethanol plants. Further study of these proteins demonstrated that these recombinant polypeptides were proteases that were specific for directly-O-linked, mannose glycosylated proteins, particularly those having such glycosylation in the linker region of modular proteins, such as those produced by yeast and fungi.

### 3. Characteristic of Proteases Specific for Mannose-Modified Proteins

The first identified protease specific for mannose-modified proteins is referred to as IFF05497 (SEQ ID NO: 1). Numerous data were collected using this molecule, particularly involving the clarification of stillage from a fuel ethanol facility and the agglomeration of yeast. Further studies revealed that IFF05497 was a protease specific for mannose-decorated amino acid sequences in the linkers of certain hydrolases, which can also be referred to as directly-O-linked, mannose glycosylated proteins.

Additional molecules that demonstrated the same modification of mannose-decorated amino acid sequences were identified by a number of methods, including sequence identity These molecules include the following, where the amino acid and nucleic acid sequences, respectively, are in parenthesis: IFF21332 (SEQ ID NO: 2), IFF21333 (SEQ ID NO: 3), IFF21334 (SEQ ID NO: 4), IFF21335 (SEQ ID NO: 5), IFF21338 (SEQ ID NO: 6), IFF21340 (SEQ ID NO: 7), IFF21347 (SEQ ID NO: 8), IFF21350 (SEQ ID NO: 9), IFF21354 (SEQ ID NO: 11), IFF21359 (SEQ ID NO: 12), IFF21360 (SEQ ID NO: 13), IFF21363 (SEQ ID NO: 15), IFF21364 (SEQ ID NO: 16), IFF21365 (SEQ ID NO: 17), IFF21372 (SEQ ID NO: 18), IFF21374 (SEQ ID NO: 19), IFF21375 (SEQ ID NO: 20), IFF21378 (SEQ ID NO: 21), IFF21379 (SEQ ID NO: 22), IFF21380 (SEQ ID NO: 23), IFF21344 (SEQ ID NO: 24), IFF21366 (SEQ ID NO: 26), IFF21331 (SEQ ID NO: 36), IFF21336 (SEQ ID NO: 37), IFF21337 (SEQ ID NO: 38), IFF21339 (SEQ ID NO: 39), IFF21341 (SEQ ID NO: 40), IFF21342 (SEQ ID NO: 41), IFF21343 (SEQ ID NO: 42), IFF21345 (SEQ ID NO: 43), IFF21346 (SEQ ID NO: 44), IFF21348 (SEQ ID NO: 45), IFF21349 (SEQ ID NO: 46), IFF21351 (SEQ ID NO: 47), IFF21352 (SEQ ID NO: 48), 21367 (SEQ ID NO: 53), IFF21368 (SEQ ID NO: 54), IFF21369 (SEQ ID NO: 55), IFF21371 (SEQ ID NO: 57) and IFF21377 (SEQ ID NO: 59). Additional molecules can be identified based on amino acid sequence identity the refined Hidden Markov Model (HMM), named “TreSub-21374_NRBlast_HSS-id35-qc70_T2k,” submitted electronically, herewith (see, e.g., Benson, G. (2011) Nuc. Acids Res., 39, pp. W29-W37 and Eddy, S.R. (2011) Accelerated Profile HMM Searches. PLOS Computational Biology 7: e1002195. https://doi.org/10.1371/journal.pcbi.1002195).

The identified proteases specific for mannose-modified proteins were readily expressed, and in some cases purified, by conventional methods. Further optimization is no doubt possible with further experimentation.

### 4. Applications for Proteases Specific for Mannose-Modified Proteins

There exist myriad heretofore unknown applications for the treatment of target mannose-modified proteins by proteases specific for mannose-decorated amino acid sequences. It is recognized that such applications must exclude those in which such target proteins and proteases are in contact in nature. No embodiment of the present compositions and methods should be construed as encompassing events that occur in nature. All embodiments of the present compositions and methods occur in a non-naturally-occurring environment, most likely in an industrial setting, which includes a pharmaceutical industrial setting, where the mannose-decorated amino acid sequences and proteases specific for mannose-modified proteins do not interact without human intervention. In some embodiments, the target mannose-modified protein and recombinant polypeptide having mannose-specific glycoprotease activity are from different organisms.

One application of present compositions and methods is the aggregation or agglomeration of yeast or fungal cells, cell bodies and/or cell components of disrupted yeast. Without being bound by theory, mannoproteins are present on hydrophilic surface on the yeast or fungal cell fragments, including the cell membrane. These surfaces becomes more hydrophobic when the mannoproteins are hydrolyzed. The more hydrophobic yeast or fungal cells or cell fragments then aggregate in an aqueous environment. Aggregated yeast or fungal cells or fragments are more easily removed from solutions and suspensions than intact yeast and fungal cells, and fragments, thereof.

In one embodiment, the compositions and methods are used to remove yeast and/or yeast components from a fermentation as in the case of beer or wine-making. Aggregated yeast and components are more easily removed from a fermentation product by filtration, centrifugation or even settling. Removal of yeast and components results in clarification of the fermentation product, which is usually desirable except in the case of certain beer styles.

In a related embodiment, the compositions and methods are used to remove yeast and/or yeast components from a fermentation in a fuel ethanol facility. This may occur prior to distillation to produce a yeast side-product useful in animal feed. This may alternatively occur following distillation to alter the characteristics of stillage products. As show in the Examples, treatment of stillage or thin stillage with proteases specific for mannose-modified proteins results in the settling of suspended solids that are rich in protein. Accordingly, solid stillage products, such as DG, DDG and DDGS have increased protein content, increasing their value as animal feed.

In another embodiment, the compositions and methods are used to remove yeast and/or yeast components following the expression of valuable proteins or small molecules other than ethanol in submerged culture. As above, aggregated yeast and components are more easily removed from a cultures by filtration, centrifugation or even settling.

In related embodiments, the compositions and methods are used to remove other fungal cells and fungal cell components with mannose-modified proteins on hydrophobic surfaces. Such cells include ascomycetes and basidiomycetes cells.

All references cited herein are herein incorporated by reference in their entirety for all purposes. To further illustrate the compositions and methods, and advantages thereof, the following specific examples are given with the understanding that they are illustrative rather than limiting.

### EXAMPLES

### Example 1: Expression and Purification of Proteins for Testing for Stillage Modification

The protein molecules to be assayed, for which the names, amino acid sequences and nucleic acid sequences are described, herein, are shown in Table 1. Gene encoding the proteins were synthesized and cloned into expression vectors using standard molecular biology procedures. Proteins were prepared as described in WO2018/005225A1.

TABLE 1
Molecule names and associated SEQ ID NOS

| Name | Amino acid SEQ ID NO: |
|---|---|
| IFF05497 | 1 |
| IFF21332 | 2 |
| IFF21333 | 3 |
| IFF21334 | 4 |
| IFF21335 | 5 |
| IFF21338 | 6 |
| IFF21340 | 7 |
| IFF21347 | 8 |
| IFF21350 | 9 |
| IFF21353 | 10 |
| IFF21354 | 11 |
| IFF21359 | 12 |
| IFF21360 | 13 |
| IFF21362 | 14 |
| IFF21363 | 15 |
| IFF21364 | 16 |
| IFF21365 | 17 |
| IFF21372 | 18 |
| IFF21374 | 19 |
| IFF21375 | 20 |
| IFF21378 | 21 |
| IFF21379 | 22 |
| IFF21380 | 23 |
| IFF21344 | 24 |
| IFF21358 | 25 |
| IFF21366 | 26 |
| IFF05588 | 27 |
| IFF07399 | 28 |
| IFF01509 | 29 |
| IFF01540 | 30 |
| IFF06679 | 31 |
| IFF03904 | 32 |
| IFF01073 | 33 |
| IFF08955 | 34 |
| IFF08955v3 | 35 |
| IFF21331 | 36 |
| IFF21336 | 37 |
| IFF21337 | 38 |
| IFF21339 | 39 |
| IFF21341 | 40 |
| IFF21342 | 41 |
| IFF21343 | 42 |
| IFF21345 | 43 |
| IFF21346 | 44 |
| IFF21348 | 45 |
| IFF21349 | 46 |
| IFF21351 | 47 |
| IFF21352 | 48 |
| IFF21355 | 49 |
| IFF21356 | 50 |
| IFF21357 | 51 |
| IFF21361 | 52 |
| IFF21367 | 53 |
| IFF21368 | 54 |
| IFF21369 | 55 |
| IFF21370 | 56 |
| IFF21371 | 57 |
| IFF21376 | 58 |
| IFF21377 | 59 |

### Example 2: Treatment of Whole Stillage Slurry

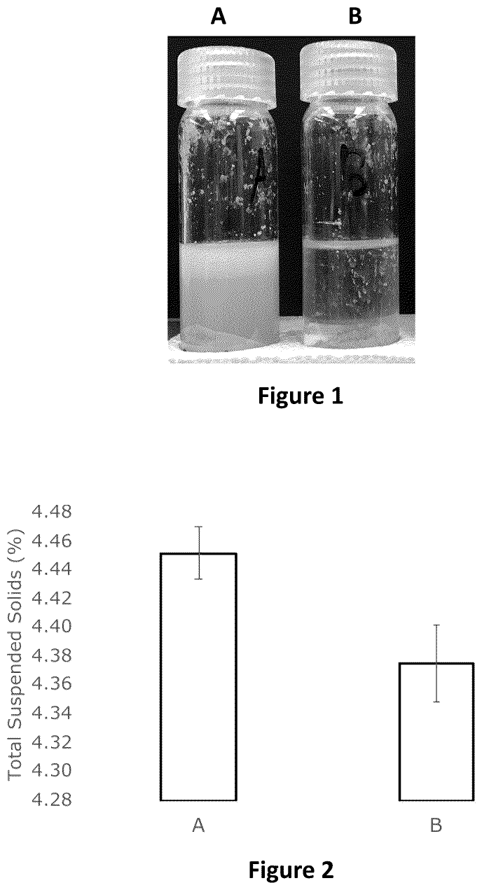

Whole stillage (20 g) was loaded into 50-mL screw-cap, round-bottom, centrifuge tubes. Sodium azide (50 μL of a 50 μg/mL solution) was added to each tube. A crude preparation of IFF05497 (110 μL for a total protein addition of 220 μg) was added to two tubes. Two tubes were retained as enzyme-free controls. A compensating volume of water (110 μL) was added to the control tubes to match the volume of enzyme added. The tubes were incubated at 32° C. for three days on a rotating mixer. The tubes were removed from incubation and centrifuged at 1,370 rcf for 30 min. As shown in FIG. 1, the supernatant of the whole stillage treated with IFF05497 had greater clarity than the supernatant of the enzyme-free control.

The reaction pellets were then suspended in 10 mL water and washed through stacked sieves with 1 mm and 200 μm mesh sizes followed by two rinses of 15 mL. Collected ultrafine solids were washed from the surfaces of the sieves with three rinses of 15 mL. Material that passed through the 200 μm mesh, considered ultrafine solids, were collected by centrifugation and dried at 65° C. over night.

The protein content of the ultrafine solids were determined using total nitrogen analysis (Costech). Treatment of whole stillage solids with a crude preparation of IFF05497 resulted in ultrafine particle solids with a greater content of total protein (Table 2), suggesting that the ultrafine particle solids were rich in protein.

TABLE 2
Fraction of protein in ultrafine fiber

| Addition | Protein (%) | sd |
|---|---|---|
| water | 52.9 | 0.7 |
| IFF05497 | 57.6 | 0.5 |

### Example 3: Suspended Solids from Whole Stillage

Whole stillage from a dry grind ethanol plant was collected and treated with or without IFF05497. 74 g of whole stillage was poured into two 125 mL Erlenmeyer flasks. To each flask was added 74 μL of a 50 g/L sodium azide stock solution (see, above). To one flask, 30.4 μL of IFF05497 (1.6 mg) was added, while 30.4 μL of water was added to the other flask. The flasks were allowed to incubate at 32° C. and 150 rpm. After 46 hours, the flasks were removed from the incubator and approximately 50 g of whole stillage from each flask was poured into 50 mL centrifuge tubes. The tubes were centrifuged for 5 minutes at 3,000 rpm. Approximately 3 g of supernatant was added to a pre-weighed tray and placed into a 70° C. oven to dry for approximately 72 hours. Afterwards, the dry tray was weighed to determine total suspended solids. Total suspended solids are reported in Table 3 and illustrated in FIG. 2. Whole stillage treated with IFF05497 showed a decrease in suspended solids.

TABLE 3
Total suspended solids of whole stillage supernatant
after treatment with or without IFF05497

| Condition | Suspended solids (%) | sd |
|---|---|---|
| No enzyme | 4.45 | 0.02 |
| IFF05497 | 4.38 | 0.03 |

### Example 4: Dynamic Light Scattering of Filtered Thin Stillage Treated with IFF05497

Thin stillage was prepared from whole stillage by centrifugation at 1,370 rcf for 10 min and further processed by filtering through a 0.45 μm syringe filter. A crude preparation of IFF05497 was diluted in buffer (50 sodium acetate, pH 5.0) and added to filtered thin stillage samples to a final concentration of approximately 6 nM, 3 nM, and 1.5 nM.

The diffusion coefficients of suspended particles in the filtered thin stillage were measured using a Wyatt dynamic light scattering microtiter plate system and Corning (3880) 96-well plates with black sides and clear, flat bottoms. Measurements were made continuously over 2.5 hr after enzyme addition.

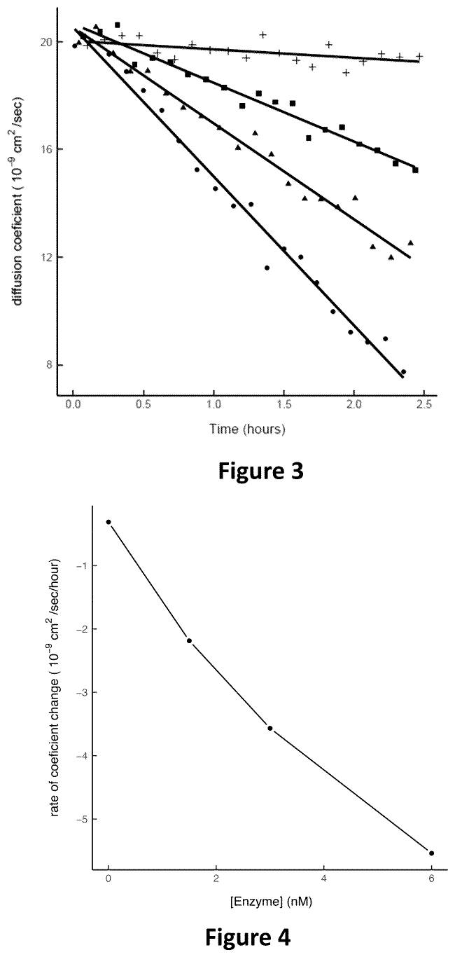

Dynamic light scattering was measured with thin stillage filtered at 0.45 μm. As shown in FIG. 3, measured values of diffusion coefficient for particles in thin stillage changed with time as a result of the addition of IFF05497.

As shown in FIG. 4, greater concentrations of IFF05497 caused a more rapid decrease in diffusion coefficients. The decrease of diffusion coefficient is consistent with the increase in particle size, which is consistent with the observed increased clarity of supernatant from whole stillage treated with IFF05497.

### Example 5: IFF05497 Treatment of Thin Stillage

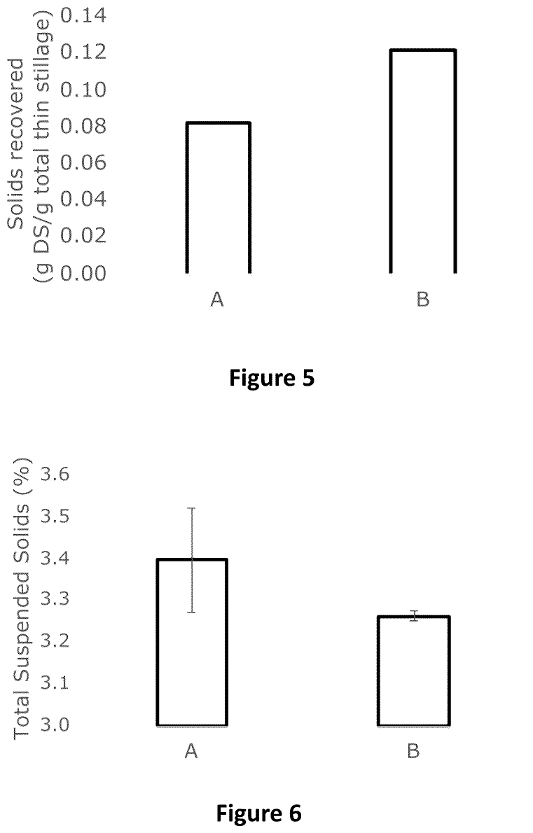

Whole stillage from a dry grind ethanol plant was collected and used to make thin stillage in the lab. As such, two 1 L bottles were filled with whole stillage and centrifuged for 5 minutes at 3,000 rpm. The supernatant was collected and was used as a thin stillage sample. 50 g of this thin stillage was weighed into two 250 mL Erlenmeyer flasks. To each flask was added 50 μL of a 50 g/L sodium azide stock solution. To one flask, 20 μL of a crude preparation of IFF05497 (1.1 mg total protein) was added. The flasks were incubated at 32° C. and 150 rpm. After 46 hr, the flasks were removed from the incubator and the entire content of each flask was poured into pre-weighed 50 mL centrifuge tubes. The tubes were centrifuged for 5 minutes at 3,000 rpm. The liquid was decanted, with approximately 3 g of supernatant transferred to a pre-weighed tray. The sample tray and centrifuge tube containing the thin stillage pellet were placed in a 70° C. oven to dry for approximately 72 hr. The dry trays and tubes were weighed to determine total suspended solids and solids recovery. The solids recovery is shown in FIG. 5 and reported in Table 4. Treating thin stillage treated with IFF05497 resulted in an increase in solids recovery. The total suspended solids is shown in FIG. 6 and reported in Table 5. Treating thin stillage treated with IFF05497 resulted in a decrease in suspended solids.

TABLE 4
Solids recovery from thin stillage after treatment
with or without IFF05497 in units of
recovered solids per total thin stillage.

| Condition | Recovered solids (%) |
|---|---|
| No enzyme | 0.083 |
| IFF05497 | 0.122 |

TABLE 5
Total suspended solids of thin stillage supernatant
after treatment with or without IFF05497.

| Condition | Suspended solids (%) | sd |
|---|---|---|
| No enzyme | 3.40 | 0.13 |
| IFF05497 | 4.265 | 0.012 |

### Example 6: Treatment of Corn Liquefact Slurry During Fermentation

A slurry of corn liquefact (35% total dry solids) was supplemented with 600 ppm urea, adjusted to a pH of 4.8 using sulfuric acid, dosed with alpha-amylase, glucoamylase and protease and dry pitched with active dry yeast at 0.1% wt/wt. The prepared slurry (100 g) was distributed into flasks. A crude preparation of IFF05497 was added to triplicate flasks at a final dosing of 7.7 μg protein/(g total dry solids), 30.7 μg protein/(g total dry solids) and 99.6 μg protein/(g total dry solids). The flasks were capped allowing for carbon dioxide release and incubated for 65 hours at 32° C.

Following incubation, 84 g slurry was filtered through a 250 μm sieve and the liquid fraction containing ultra fine particles was collected with the application of gentle vacuum pressure. Fiber cake was transferred from the surface of the sieve to a wash beaker and suspended in 90 mL water. The fiber was returned to the sieve and the liquid fraction again collected. The transfer, suspension and collection steps were repeated for a total of four liquid fraction collections. Ultrafine fiber material was collected from the liquid fraction by centrifugation at 1,370 rcf. Supernatant was removed by aspiration. Ultrafine fiber material from individual fractions were resuspended in water and combined into a single tube. The final sample of ultrafine fiber material was collected by centrifugation at 1,370 rcf and wash-water supernatant was removed by aspiration. Ultrafine fiber material was dried for 3 days at 65° C. The protein content of the ultrafine solids were determined using total nitrogen analysis (Costech). As shown in Table 6, the addition of IFF05497 before fermentation results in an increase of protein content recovered from the ultrafine material.

TABLE 6
Fraction of protein in ultrafine fiber from
SSF (%) as a result of incubation with
IFF05497 (dose is reported in units
of μg protein/(g total dry solids)).

| Enzyme dose | | |
|---|---|---|
| (μg/g) | Protein (%) | sd |
| water | 52.1 | 0.4 |
| 7.7 | 52.5 | 0.6 |
| 33.7 | 52.9 | 0.4 |
| 99.6 | 52.9 | 0.3 |

### Example 7: IFF05497 Treatment of Budding Yeast

A conventional strain of Saccharomyces cerevisae well-known in the grain ethanol industry was propagated in a solution of yeast extract, peptone and dextrose (YPD) in the presence or absence of IFF05497. Briefly, active dry yeast (ADY; Ethanol Red) was added at 0.1% w/w to six identical baffled flasks containing 100 g of a mixture of YPD containing 20% glucose and 600 ppm urea. Three of the flasks were further supplemented with 37.6 μL of IFF05497 (2.03 mg total protein) while the others received 37.6 μL of water. The flasks were allowed to incubate at 200 rpm at 32° C. for 26 hours. Following incubation, the content of each flask was distributed across two 50 mL centrifuge tubes and centrifuged at 3,000 rpm for 10 min.

The supernatant was decanted, and the resulting yeast pellets were washed with water and subjected to repeated centrifugation and decanting. The resulting washed pellet was allowed to dry in an oven at 70° C. for approximately 72 hr. The dried yeast pellets were milled using an IKA tube mill 100. The resulting dried and milled yeast powder was pooled from the triplicate samples into duplicate samples for protein determination by combustion and nitrogen measurement. The protein content (calculated from the measured nitrogen content) is shown in Table 7 for the yeast pellets with or without IFF05497 treatment. The protein content of the yeast grown in the presence IFF05497 was higher (an average of 54.2% protein) than the yeast that was not subjected to any enzyme treatment (53.1% protein).

TABLE 7
Protein content of residual yeast grown in
ADY with or without IFF05497

| | Average protein content of |
|---|---|
| Conditions | residual yeast pellet (%) |
| No enzyme replicate 1 | 53.1 |
| No enzyme replicate 2 | 53.0 |
| IFF05497 replicate 1 | 54.2 |
| IFF05497 replicate 2 | 54.2 |

### Example 8: Protein Content of Yeast after IFF05497 Treatment

A strain of Saccharomyces cerevisae was propagated in YPD as above. The flasks were incubated at 150 rpm at 32° C. for 21 hr. Following incubation, the flask contents were poured into six 50 mL centrifuge tubes and centrifuged at 3,000 rpm for 5 min. The supernatant was decanted, and the resulting yeast pellets were washed with Milli-Q water by repeated centrifugation and decanting. 2.5 mL of water and 2.5 mL of 0.3 sodium acetate buffer (pH 5.3) was then added to each tube. The yeast pellets were slurried by vortexing and collected together in one beaker.

The beaker was then placed onto a stir plate with a stir bar. While stirring, 10 mL of the prepared yeast slurry was pipetted into four 20 mL glass scintillation vials. To each vial, sodium azide was added to a final concentration of 0.17%. To two of the vials, 5 μL of a crude preparation of IFF05497 (0.27 mg total protein) was added. Vials were capped and incubated at 150 rpm at 32° C.

After 26 hours, the vials were removed, and samples were collected in 50 mL centrifuge tubes. The tubes were centrifuged at 3,000 rpm for 5 minutes. A portion (1 mL) of the supernatant was collected for liquid analysis, as described below. The remaining supernatant was decanted and the pellet was washed with water by repeated centrifugation and decanting. The resulting washed pellet was allowed to dry in an oven at 70° C. for approximately 72 hr.

The dried yeast pellet was milled using an IKA tube mill 100. The resulting dried and milled yeast powder was pooled for the duplicate samples for protein determination by combustion and nitrogen measurement. The protein content, calculated from the measured nitrogen content, for the yeast pellet with or without IFF05497 treatment is shown in Table 8. The protein content of the yeast pellet subjected to IFF05497 treatment was higher (60.1% protein) than the yeast pellet that was not subjected to any enzyme treatment (57.8% protein).

TABLE 8
Protein content of residual yeast after
treatment with or without IFF05497

| | Protein content of |
|---|---|
| Conditions | residual yeast pellet (%) |
| No enzyme | 57.8 |
| IFF05497 | 60.1 |

The liquid samples collected after centrifugation of the treated yeast samples were filtered through a 0.22 μm spin filter. The resulting filtered liquid was injected directly into an Agilent high performance liquid chromatography (HPLC) instrument equipped with an refractive index (RI) detector and Phenomenex Rezex Organic Acids H+ ROA 150×7.8 mm column at 80° C., running an isocratic mobile phase of 0.01 sulfuric acid at 0.6 mL/min.

The filtered liquid samples were also subjected to acid hydrolysis to determine the total sugar content (monomer and oligomer). 50 μL of filtered sample was mixed with 50 μL of 0.8 sulfuric acid and placed in a pressure sealed 96-well plate. The plate was placed in an autoclave and heated at 121° C. for 45 minutes. After allowing to cool, 50 μL of water was added to each sample, and the resulting mixed samples were injected onto an HPLC as described previously.

The peak appearing at 5.07 minutes is reasonably presumed to be mannose. Mannose was not detected in samples that were directly injected with no acid treatment, indicating that IFF05497 did not release any monomer mannose. Accordingly, the amount of mannose detected in acid treated samples represented manno-oligomers, as summarized in Table 9. As such, the yeast sample treated with IFF05497 released 5.9 times more manno-oligomers than the no enzyme control.

TABLE 9
Manno−oligomer content in filtered yeast
hydrolysate after treatment with or without

| | Manno−oligomer concentration in |
|---|---|
| Condition | yeast hydrolysate (mg L⁻¹) |
| No enzyme | 33.4 |
| IFF05497 | 197.3 |

### Example 9: Incubation of IFF05497 with Yeast Causes Aggregation

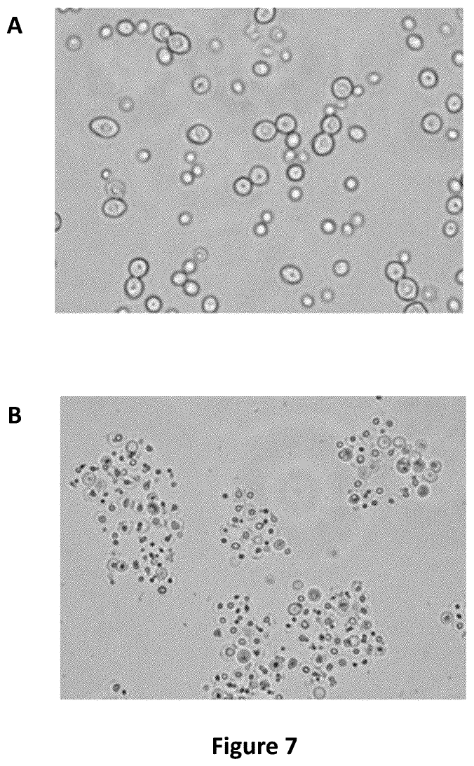

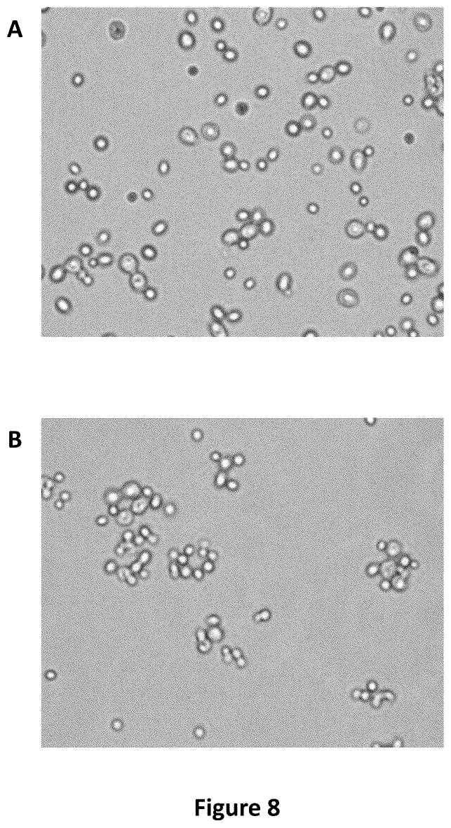

Purified IFF05497 (10 μL of 2 mg/mL) was combined with active yeast (500 μL of 1% wt/wt suspension) and incubated at 32° C. for 24 hours. Yeast samples were diluted 100-fold in water and observed by microscopy. Yeast cells treated with IFF05497 were found aggregated (FIG. 7B), while untreated cells were dispersed, FIG. 7A.

Yeast was inactivated by heat treatment at 95° C. for 30 min. Purified IFF05497 (10 μL of 2 mg/mL) was combined with inactivated yeast (500 μL of 1% wt/wt suspension) and incubated at 32° C. for 24 hours. Yeast samples were diluted 100-fold in water and observed by microscopy. Inactivated yeast cells treated with IFF05497 were again found aggregated (FIG. 8B), while untreated cells were dispersed FIG. 8A).

### Example 10: IFF05497 Catalytic Activity and Specificity

Purified IFF05497 was combined in a ratio of 1 part with 20 parts purified target proteins, i.e., IFF05588, IFF07399, IFF01509, IFF01540, IFF06679, IFF03904, IFF08955, IFF08955v3 (which includes an artificial linker) or IFF01073 in 20 mM sodium acetate at pH 5.0. Reactions were incubated overnight at 35° C. Features of the target proteins are summarized in Table 10. Reactions were combined with SDS-PAGE loading dye and 5 μg total protein were loaded in the wells of an SDS-PAGE gel.

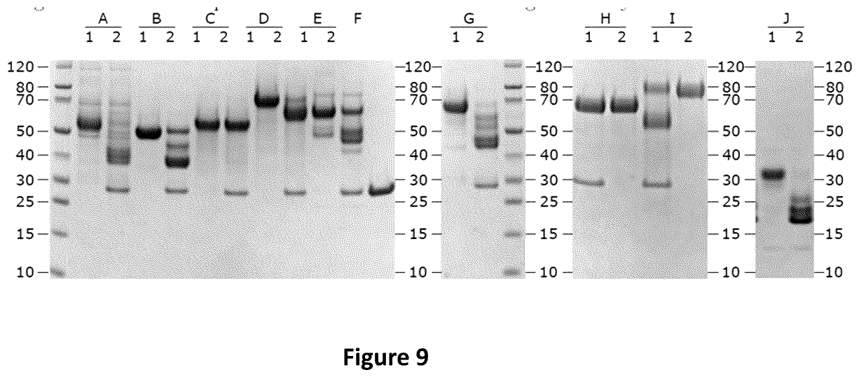

Proteins containing a binding module, linker and core domains were altered by incubation with IFF05497 resulting in a mobility shift (FIG. 9). Lane assignments are summarized in Table 10. These results indicate that IFF05497 is a protease specific for protein substrates with the presence of a linker between a binding module and core domain. In this example, the binding module is a carbohydrate-binding module (CBM).

TABLE 10
Protein samples treated with IFF05497 show gel mobility shift.

| | | | Mobility |
|---|---|---|---|
| Lane | Protein | Domain structure | change |
| A1 | IFF05588 | CBM-linker-core | — |
| A2 | IFF05588 and IFF05497 | CBM-linker-core | yes |
| B1 | IFF07399 | CBM-linker-core | — |
| B2 | IFF07399 and IFF05497 | CBM-linker-core | yes |
| C1 | IFF01509 | core | — |
| C2 | IFF01509 and IFF05497 | core | no |
| D1 | IFF01540 | core-linker-CBM | — |
| D2 | IFF01540 and IFF05497 | core-linker-CBM | yes |
| E1 | IFF06679 | CBM-linker-core | — |
| E2 | IFF06679 and IFF05497 | CBM-linker-core | yes |
| F | IFF05497 | | |
| G1 | IFF03904 | CBM-linker-core | — |
| G2 | IFF03904 and IFF05497 | CBM-linker-core | yes |
| H1 | IFF08955 | core-CBM | — |
| H2 | IFF08955 and IFF05497 | core-CBM | no |
| I1 | IFF08955v3 | core-linker-CBM | — |
| I2 | IFF08955v3 and IFF05497 | core-linker-CBM | yes |
| J1 | IFF01073 | core-linker-CBM | — |
| J2 | IFF01073 and IFF05497 | core-linker-CBM | yes |

Mass spec peptide analysis of samples of IFF07399 treated by IFF05497 then treated with trypsin identified peptides from IFF07399 with hexose modifications (Table 11). Numbers in subscript indicate the amino acid positions in the sequence of IFF07399. The number of hexose modifications detected are indicated for each peptide sequence identified.

TABLE 11
Peptides identified after treatment of IFF07399
by IFF05497.

| Peptide sequence | Hexose residues determined |
|---|---|
| T₄₉LKTTTT₅₅ | 9 |
| T₅₆SSTSSAPTGK₆₆ | 5, 6, 7 and 8 |
| S₅₇STSSAPTGK₆₆ | 5 |
| S₅₈TSSAPTGK₆₆ | 4 |
| T₅₉SSAPTGK₆₆ | 4 |

Proteins expressed in T. reesei and other fungi are known to be modified with mannose sugars on some threonine and some serine residues, especially in linker domains. Up to three mannose units are found attached to a single serine or a single threonine. IFF05497 appears to be a protease specific for cleavage before a mannose-modified serine or mannose modified threonine.

### Example 11: Target Proteins with and without O-Glycosylation

IFF01073 was produced in E. coli without glycosylation and also in T. reesei with glycosylation. Purified IFF05497 was combined in a ratio of 1 part with 20 parts of a crude preparation of IFF01073 expressed in E. coli or expressed in T. reesei in 20 sodium acetate pH 5.0. Reactions were incubated overnight at 35° C. Reactions were analyzed by SDS-PAGE as before.

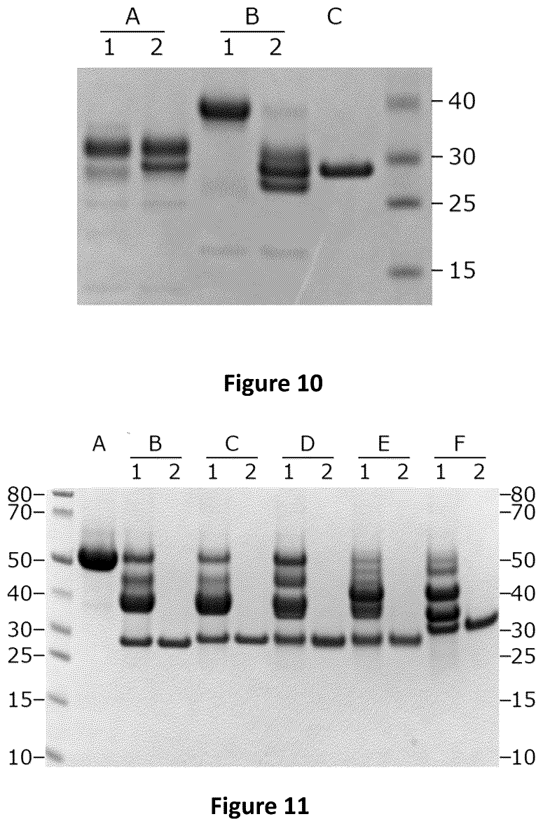

IFF05497 modified the protein IFF01073 produced in T. reesei but did not modify the same amino acid sequence produced in E. coli, which was not modified with mannose (FIGS. 10 and 11). These results confirm that IFF05497 is a protease specific for protein substrates with a directly-linked mannose modification.

TABLE 12
Activity of IFF05497 against IFF01073
produced in T. reesei or E. coli.

| | | Expression | Gel mobility |
|---|---|---|---|
| Lane | Protein | host | change |
| A1 | IFF01073 | E. coli | — |
| A2 | IFF01073 and IFF05497 | E. coli | no |
| B1 | IFF01073 | T. reesei | — |
| B2 | IFF01073 and IFF05497 | T. reesei | yes |
| C | IFF05497 | T. reesei | — |

### Example 12: Activity of Proteins Related to IFF05497

Purified IFF05497, IFF21344, IFF21358, IFF21366 or IFF21374 were combined in a ratio of 1 part with 20 parts IFF07399 in 20 sodium acetate pH 5.0. Reactions were incubated overnight at 35° C. Reactions were analyzed by SDS-PAGE as before.

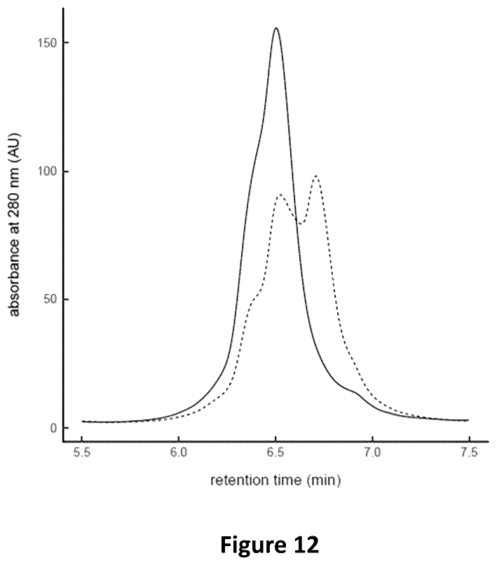

IFF05497, IFF21344, IFF21358, IFF21366, and IFF21374 all have the ability to cause a mobility shift of the protein IFF07399 (FIGS. 11 and 12 and Table 13). All appear to be proteases with activities and specificities similar to IFF05497.

TABLE 13
IFF07399 incubated with IFF05497 or related proteins

| Lane | Protein | Gel mobility change |
|---|---|---|
| A1 | IFF07399 | — |
| B1 | IFF07399 and IFF05497 | yes |
| B2 | IFF05497 | — |
| C1 | IFF07399 and IFF21344 | yes |
| C2 | IFF21344 | — |
| D1 | IFF07399 and IFF21358 | yes |
| D2 | IFF21358 | — |
| E1 | IFF07399 and IFF21366 | yes |
| E2 | IFF21366 | — |
| F1 | IFF07399 and IFF21374 | yes |
| F2 | IFF21374 | — |

Crude samples of proteins related to IFF05497 (i.e., IFF21359, IFF21354, IFF21335, IFF21360, IFF21334, IFF21332, IFF21375, IFF21350, IFF21365, IFF21380, IFF21338, IFF21372, IFF21333, IFF21347, IFF21378, IFF21374, IFF21364, IFF21379, IFF21340, IFF21363, IFF21353, and IFF21362) were prepared by fermentation at shake flask scale. Supernatants from the fermentations were concentrated 10-fold using a centrifugal protein concentration device with 5 kDa nominal molecular weight cut-off to a final concentration of 1.8 g L⁻¹ to 5.5 g L⁻¹. Concentrated samples were buffer-exchanged into 50 sodium acetate buffer at pH 5.0 using size-exclusion chromatography resin with a 7 kDa nominal molecular weight cut-off. A crude preparation of IFF05588 was diluted to a final concentration of 2 g/L in 50 sodium acetate buffer at pH 5.0.

The concentrated preparations of IFF05497-related crude proteins (5 μL with concentrations ranging from 1.8 g L⁻¹ to 5.5 g L⁻¹) were combined with the diluted preparation of IFF05588 (95 μL of 2 g/L dilution). Reactions were incubated at 25° C. for 24 hours. The samples were then filtered and analyzed by reverse phase chromatography using a Zorbax 300SB-C3 column with a gradient of water and acetonitrile with 0.1% trifluoroacetic acid.

Modification of IFF05588 was observed as the development of a peak with a later retention time than the peaks obtained from intact IFF05588 (FIG. 12). The total peak area of IFF05588 and the peak area after 6.6 minutes were quantitated and a ratio of late peak area divided by the total peak area was calculated to represent the extent of modification of IFF05588. This ratio is a relative late peak area and is reported in Table 14. All samples containing proteins related to IFF05497 except one (IFF21362) are able to modify IFF05588 under these reaction conditions.

TABLE 14
IFF05588 incubated with IFF05497 or related proteins

| Protease | Relative late peak area |
|---|---|
| IFF21359 | 0.68 |
| IFF21354 | 0.67 |
| IFF21335 | 0.66 |
| IFF21360 | 0.66 |
| IFF21334 | 0.65 |
| IFF21332 | 0.65 |
| IFF21375 | 0.63 |
| IFF21350 | 0.61 |
| IFF21365 | 0.61 |
| IFF21380 | 0.61 |
| IFF21338 | 0.60 |
| IFF21372 | 0.60 |
| IFF21333 | 0.59 |
| IFF21347 | 0.59 |
| IFF21378 | 0.52 |
| IFF21374 | 0.51 |
| IFF21364 | 0.49 |
| IFF21379 | 0.45 |
| IFF21340 | 0.33 |
| IFF21363 | 0.30 |
| IFF21353 | 0.25 |
| Buffer | 0.24 |
| IFF21362 | 0.23 |

### Example 13: Further Activity of Proteins Related to IFF05497

Crude samples of proteins related to IFF05497 (IFF21331, IFF21332, IFF21333, IFF21334, IFF21335, IFF21336, IFF21337, IFF21338, IFF21339, IFF21340, IFF21341, IFF21342, IFF21343, IFF21344, IFF21345, IFF21346, IFF21347, IFF21348, IFF21349, IFF21350, IFF21351, IFF21352, IFF21353, IFF21354, IFF21355, IFF21356, IFF21357, IFF21358, IFF21359, IFF21360, IFF21361, IFF21362, IFF21363, IFF21364, IFF21365, IFF21366, IFF21367, IFF21368, IFF21369, IFF21370, IFF21371, IFF21372, IFF21374, IFF21375, IFF21376, IFF21377, IFF21378, IFF21379, and IFF21380) were prepared by fermentation in shake flasks to final concentrations of 0.8 g/L to 2.6 g/L. A dilution series of enriched IFF05497 was prepared starting at 5.9 g/L and serially diluted 10-fold, 100-fold and 1,000-fold and 10,000-fold for inclusion with the crude samples in reaction testing. A crude preparation of IFF05588 was diluted to a final concentration of 2 g/L in 50 sodium acetate buffer at pH 5.0.

The crude preparations of IFF05497-related proteins (5 μL) were combined with the diluted preparation of IFF05588 (95 μL). Reactions were incubated at 25° C. for 5, 10 and 15 minutes before combining with EDTA (0.5 M). The samples were then filtered and analyzed by reverse phase chromatography using a Zorbax 300SB-C3 column with a gradient of water and acetonitrile with 0.1% trifluoroacetic acid.

Modification of IFF05588 was observed as the development of a peak with a later retention time than the peak of the intact IFF05588 as demonstrated in FIG. 12 showing treatment of IFF05588 with IFF21347.

The total peak area of IFF05588 and the peak area after 6.6 minutes were quantitated and a ratio of late peak area divided by the total peak area was calculated to represent the extent of modification of IFF05588. The relative peak area values for each timepoint were centered by subtracting the average value of all relative peak areas observed at that timepoint. Those centered difference values in peak area were scaled by dividing the centered difference values by the standard deviation of relative peak areas. The centered and scaled value for relative peak areas at all timepoints were then averaged for each sample. This averaged scaled and centered ratio is a representation of relative activity and is reported in Table 15. All samples containing proteins related to IFF05497 except seven (IFF21355, IFF21358, IFF21356, IFF21361, IFF21370, IFF21357 and IFF21353) are able to modify IFF05588 under these reaction conditions.

TABLE 15
IFF05588 incubated with IFF05497 or related proteins
showing a shift of retention time and peak shape

| Protease | Relative late peak area |
|---|---|
| IFF21345 | 1.82 |
| IFF21347 | 1.74 |
| IFF21372 | 1.62 |
| IFF21350 | 1.55 |
| IFF21348 | 1.40 |
| IFF21334 | 1.38 |
| IFF21344 | 1.36 |
| IFF21354 | 1.34 |
| IFF21359 | 1.30 |
| IFF21338 | 1.11 |
| IFF21375 | 1.08 |
| IFF21365 | 1.04 |
| IFF21341 | 1.03 |
| IFF21337 | 0.90 |
| IFF21335 | 0.67 |
| IFF21346 | 0.52 |
| IFF21366 | 0.34 |
| IFF21343 | 0.33 |
| IFF21332 | 0.30 |
| IFF21333 | 0.26 |
| IFF21342 | 0.12 |
| IFF21349 | 0.02 |
| IFF21379 | −0.02 |
| IFF21339 | −0.05 |
| IFF21340 | −0.31 |
| IFF21336 | −0.34 |
| IFF21363 | −0.43 |
| IFF21364 | −0.57 |
| IFF21351 | −0.57 |
| IFF21360 | −0.58 |
| IFF21378 | −0.60 |
| IFF21352 | −0.63 |
| IFF21367 | −0.67 |
| IFF21380 | −0.71 |
| IFF05497 | −0.77 |
| IFF21331 | −0.86 |
| IFF21371 | −0.95 |
| IFF21377 | −0.99 |
| IFF21362 | −0.99 |
| IFF21376 | −0.99 |
| IFF21368 | −1.00 |
| IFF21369 | −1.00 |
| buffer | −1.02 |
| IFF21355 | −1.02 |
| IFF21358 | −1.04 |
| IFF21356 | −1.04 |
| IFF21361 | −1.05 |
| IFF21370 | −1.05 |
| IFF21357 | −1.05 |
| IFF21353 | −1.06 |

Some crude samples prepared for short time reaction incubations were further diluted 2-fold or 10-fold, depending on activity level observed in short time incubation. Some samples were used without further dilution (see Table 16). The diluted crude samples and controls were then combined (1 μL, 2 μL, and 5 μL) with the diluted preparation of IFF05588 (95 μL). Reactions were incubated at 25° C. for 24 hours before combining with EDTA (0.5 M). The samples were then filtered and analyzed by reverse phase chromatography using a Zorbax 300SB-C3 column with a gradient of water and acetonitrile with 0.1% trifluoroacetic acid. Modification of IFF05588 was observed as the development of a peak with a later retention time than the peak of the intact IFF05588 as demonstrated in FIG. 12 showing treatment of IFF05588 with IFF21347.

The total peak area of IFF05588 and the peak area after 6.6 minutes were quantitated and a ratio of late peak area divided by the total peak area was calculated to represent the extent of modification of IFF05588. The relative peak area values for each dose were centered by subtracting the average value of all relative peak areas observed at that dose. Those centered difference values in peak area were scaled by dividing the centered difference values by the standard deviation of relative peak areas. The centered and scaled value for relative peak areas at all doses were then averaged for each sample. This averaged scaled and centered ratio is a representation of relative activity and is reported in Table 16. All samples containing proteins related to IFF05497 except two (IFF21376 and IFF21361) are able to modify IFF05588 under these reaction conditions. Close comparison of the chromatograms for these two samples show differences between buffer and sample chromatograms indicative of activity in the samples.

TABLE 16
IFF05588 incubated with different doses
of IFF05497 or related proteins

| Protease | Dilution (fold) | Relative late peak area |
|---|---|---|
| IFF21345 | 10 | 1.24 |
| IFF21372 | 10 | 1.18 |
| IFF21347 | 10 | 1.14 |
| IFF21346 | 2 | 1.07 |
| IFF21350 | 10 | 1.06 |
| IFF21366 | 2 | 1.02 |
| IFF21334 | 10 | 0.97 |
| IFF21354 | 10 | 0.87 |
| IFF21343 | 2 | 0.80 |
| IFF21332 | 2 | 0.80 |
| IFF21344 | 10 | 0.79 |
| IFF21379 | 2 | 0.75 |
| IFF21342 | 2 | 0.73 |
| IFF21333 | 2 | 0.69 |
| IFF21348 | 10 | 0.68 |
| IFF21351 | 1 | 0.66 |
| IFF21341 | 10 | 0.62 |
| IFF21360 | 1 | 0.62 |
| IFF21359 | 10 | 0.61 |
| IFF21338 | 10 | 0.59 |
| IFF21349 | 2 | 0.56 |
| IFF21339 | 2 | 0.53 |
| IFF21337 | 10 | 0.52 |
| IFF21352 | 1 | 0.42 |
| IFF21375 | 10 | 0.42 |
| IFF21365 | 10 | 0.38 |
| IFF21340 | 2 | 0.37 |
| IFF21336 | 2 | 0.36 |
| IFF21335 | 10 | 0.30 |
| IFF21367 | 1 | 0.19 |
| IFF21378 | 1 | 0.10 |
| IFF21380 | 1 | −0.02 |
| IFF21363 | 2 | −0.13 |
| IFF21331 | 1 | −0.21 |
| IFF05497 | 1 | −0.28 |
| IFF21364 | 2 | −0.28 |
| IFF21371 | 1 | −1.12 |
| IFF21353 | 1 | −1.31 |
| IFF21370 | 1 | −1.33 |
| IFF21369 | 1 | −1.42 |
| IFF21368 | 1 | −1.44 |
| IFF21377 | 1 | −1.45 |
| IFF21358 | 1 | −1.46 |
| IFF21357 | 1 | −1.49 |
| IFF21362 | 1 | −1.52 |
| IFF21356 | 1 | −1.52 |
| IFF21355 | 1 | −1.52 |
| Buffer | 1 | −1.59 |
| IFF21376 | 1 | −1.61 |
| IFF21361 | 1 | −1.64 |

What is claimed is:

1. A method for modifying a mannose-decorated amino acid sequence present in a target protein comprising contacting the protein with a recombinant polypeptide having mannose-specific glycoprotease activity, wherein the contacting occurs in a non-naturally occurring environment.

2. The method of claim 1, wherein the modification is proteolysis.

3. The method of claim 1 or 2, wherein the mannose-decorated amino acid sequence is in the linker region of a target protein.

4. The method of any of the preceding claims, wherein the target protein is present on a hydrophobic surface of yeast or fungal cells, cell bodies or cellular components.

5. The method of claim 4, wherein the contacting results in aggregation of the cells, cell bodies or cellular components.

6. The method of any of the preceding claims, wherein the contacting occurs in an industrial or pharmaceutical reaction vessel.

7. The method of any of the preceding claims, wherein the target protein and recombinant polypeptide having mannose-specific glycoprotease activity are from different organisms.

8. A method for agglomerating organisms displaying mannose-decorated amino acid sequences on their surface comprising contacting the organisms with a recombinant polypeptide having mannose-specific glycoprotease activity.

9. The method of claim 8, wherein the organism is yeast or fungi.

10. The method of claim 9, wherein the organism is a Saccharomyces sp.

11. A method for modifying a fermentation product produced by yeasts cells and comprising yeast cells, cell bodies and/or cell components, comprising contacting the fermentation product with a recombinant polypeptide having mannose-specific glycoprotease activity to produce a modified fermentation product having dissolved solids with reduced optical density and/or an insoluble fraction enriched for protein.

12. The method of claim 11, wherein the reduced optical density results from aggregation of the yeast cells, cell bodies and/or cell components.

13. The method of claim 11 or 12, wherein the fermentation product is stillage from an ethanol fermentation process.

14. The method of any of the preceding claims, wherein the recombinant polypeptide having mannose-specific glycoprotease activity complies with the Hidden Markov Model TreSub-21374_NRBlast_HSS-id35-qc70_T2k.

15. The method of any of the preceding claims, wherein the recombinant polypeptide having mannose-specific glycoprotease activity has at least 90% amino acid sequence identity to an amino acid sequence selected from the group consisting of SEQ ID NO: 1 (IFF05497), SEQ ID NO: 2 (IFF21332), SEQ ID NO: 3 (IFF21333), SEQ ID NO: 4 (IFF21334), SEQ ID NO: 5 (IFF21335), SEQ ID NO: 6 (IFF21338), SEQ ID NO: 7 (IFF21340), SEQ ID NO: 8 (IFF21347), SEQ ID NO: 9 (IFF21350), SEQ ID NO: 11 (IFF21354), SEQ ID NO: 12 (IFF21359), SEQ ID NO: 13 (IFF21360), SEQ ID NO: 15 (IFF21363), SEQ ID NO: 16 (IFF21364), SEQ ID NO: 17 (IFF21365), SEQ ID NO: 18 (IFF21372), SEQ ID NO: 19 (IFF21374), SEQ ID NO: 20 (IFF21375), SEQ ID NO: 21 (IFF21378), SEQ ID NO: 22 (IFF21379), SEQ ID NO: 23 (IFF21380), SEQ ID NO: 24 (IFF21344), SEQ ID NO: 26 (IFF21366), SEQ ID NO: 36 (IFF21331), SEQ ID NO: 37 (IFF21336), SEQ ID NO: 38 (IFF21337), SEQ ID NO: 39 (IFF21339), SEQ ID NO: 40 (IFF21341), SEQ ID NO: 41 IFF21342), SEQ ID NO: 42 (IFF21343), SEQ ID NO: 43 (IFF21345), SEQ ID NO: 44 (IFF21346), SEQ ID NO: 45 (IFF21348), SEQ ID NO: 46 (IFF21349), SEQ ID NO: 47 (IFF21351), SEQ ID NO: 48 (IFF21352), SEQ ID NO: 53 (IFF21367), SEQ ID NO: 54 (IFF21368), SEQ ID NO: 55 (IFF21369), SEQ ID NO: 57 (IFF21371), and SEQ ID NO: 59 (IFF21377).

15. A stillage product obtained by the method of claim 13.

16. A recombinant polypeptide having mannose-specific glycoprotease activity and:
(a) having at least 90% amino acid sequence identity to an amino acid sequence selected from the group consisting of SEQ ID NO: 1 (IFF05497), SEQ ID NO: 2 (IFF21332), SEQ ID NO: 3 (IFF21333), SEQ ID NO: 4 (IFF21334), SEQ ID NO: 5 (IFF21335), SEQ ID NO: 6 (IFF21338), SEQ ID NO: 7 (IFF21340), SEQ ID NO: 8 (IFF21347), SEQ ID NO: 9 (IFF21350), SEQ ID NO: 11 (IFF21354), SEQ ID NO: 12 (IFF21359), SEQ ID NO: 13 (IFF21360), SEQ ID NO: 15 (IFF21363), SEQ ID NO: 16 (IFF21364), SEQ ID NO: 17 (IFF21365), SEQ ID NO: 18 (IFF21372), SEQ ID NO: 19 (IFF21374), SEQ ID NO: 20 (IFF21375), SEQ ID NO: 21 (IFF21378), SEQ ID NO: 22 (IFF21379), SEQ ID NO: 23 (IFF21380), SEQ ID NO: 24 (IFF21344), SEQ ID NO: 26 (IFF21366), SEQ ID NO: 36 (IFF21331), SEQ ID NO: 37 (IFF21336), SEQ ID NO: 38 (IFF21337), SEQ ID NO: 39 (IFF21339), SEQ ID NO: 40 (IFF21341), SEQ ID NO: 41 IFF21342), SEQ ID NO: 42 (IFF21343), SEQ ID NO: 43 (IFF21345), SEQ ID NO: 44 (IFF21346), SEQ ID NO: 45 (IFF21348), SEQ ID NO: 46 (IFF21349), SEQ ID NO: 47 (IFF21351), SEQ ID NO: 48 (IFF21352), SEQ ID NO: 53 (IFF21367), SEQ ID NO: 54 (IFF21368), SEQ ID NO: 55 (IFF21369), SEQ ID NO: 57 (IFF21371), and SEQ ID NO: 59 (IFF21377), and/or
(b) being identifiable by the Hidden Markov Model TreSub-21374_NRBlast_HSS-id35-qc70_T2k.
